(19) United States
(12) Patent Application Publication
(10) Pub. No.: US 2005/0021241 A1
(43) Pub. Date: Jan. 27, 2005

(54) Gain of function sorting for drug discovery and development

(76) Inventors: Juan A. Ballesteros, San Diego, CA (US); Teresa A. Bennett, San Diego, CA (US); Alex Okun, San Diego, CA (US); Andrew Beernink, San Diego, CA (US); John T. Ransom, Encinitas, CA (US)

Correspondence Address: PILLSBURY WINTHROP LLP, ATTENTION: DOCKETING DEPARTMENT, 11682 EL CAMINO REAL, SUITE 200, SAN DIEGO, CA, 92130, US

(21) Appl. No.: 10/841,974

(22) Filed: May 7, 2004

Related U.S. Application Data
(60) Provisional application No. 60/468,784, filed on May 7, 2003.

(51) Int. Cl. G01N 33/48

(52) U.S. Cl. 702/21

(57) ABSTRACT

The present disclosure provides compositions and methods for high throughput Gain of Function (GOF) sorting to discover and develop novel and highly selective candidate drug molecules. High throughput GOF sorting includes: mutating a single residue in a receptor and/or ligand; measuring the affinity and functional activity of the resulting ligand target-ligand interaction; and carrying out multiple rounds of mutation and measurement to determine which residues provide key interaction points underlying the functional activity of a ligand target-ligand interaction. Further, the present disclosure provides methods and compositions for high throughput and high precision GOF sorting, such that large numbers of mutations can be generated and screened rapidly, and GOF compounds can be identified and isolated.

### FIELD OF THE INVENTION

The present invention relates to methods and compositions for drug discovery by gain of function (GOF) sorting. In particular, the present invention relates to altering receptors and/or ligands, measuring ligand target-ligand interactions to identify ligand target-ligand pairs showing gain of function (GOF) relative to other ligand target-ligand pairs, and high-throughput GOF sorting. Ligands identified by GOF sorting are tested as candidate drug molecules and/or undergo additional GOF sorting.

### BACKGROUND OF THE INVENTION

Ligand target-ligand binding is a key control point for modulating cellular function and is therefore an important target for drug discovery and development. Of particular interest are G-protein-coupled receptors (GPCRs) that bind natural agonistic ligands (e.g., β-adrenergic receptor that binds norepinephrine) through specific physical and chemical interactions between the ligand and receptor. The contact sites between ligand and receptor are defined by specific amino acid residues in the receptor, their position within the three-dimensional receptor structure, and their chemical properties. These properties define the “binding pocket” within the receptor structure that accommodates the natural ligand in such a way that ligand binding in the pocket activates the receptor. Residues in the ligand interact with the critical binding pocket residues to achieve a binding interaction characterized by a relatively low free energy of interaction. Ligand binding commonly causes a conformational change within the receptor, often in the transmembrane structure, that leads to interaction of the intracellular or cytosolic domains of the receptor with second messenger structures responsible for transmitting information regarding the extracellular ligand-receptor interaction to the cell interior.

Ligand target-ligand binding leads to conformational changes in the receptor, transmembrane signal transduction, and interaction with the appropriate intracellular second messenger systems, resulting in cellular and physiological responses such as enzyme activity, ion fluxes, gene regulation, secretion, growth, movement, or contraction. The specific physical and chemical properties of the natural ligand-receptor pair provide specificity and selectivity to insure that other receptors intended for different functions do not cross-react with the incorrect ligands.

In receptors having seven transmembrane spanning regions (7 TM receptors), ligand binding often causes a conformational change in the 7 TM region that affects the intracellular or cytosolic domains of the receptor. In GPCRS, ligand binding usually causes a conformational change that causes the GPCR to interact with the appropriate G-protein to trigger the intracellular second messenger response(s) specific for that ligand.

Information about the physical and chemcial interactions between receptors and ligands is of fundamental importance to research in drug discovery. Mutation of both the receptor and the ligand provide useful information for drug discovery and development, but traditional mutational approaches have been laborious and time-consuming. Screening of drug candidates can be random. Thus, drug discovery based on the study of ligand target-ligand interaction has been hampered both by constraints on the ability to generate and test large numbers of samples, and have also been hampered by approaches to experimental design that retard progress.

### BRIEF DESCRIPTION OF THE DRAWINGS

FIG. 1 is a simplified block diagram illustrating functional components of one embodiment of a sample analysis system incorporating elements of a direct sample injection system.

FIG. 2 is a simplified block diagram illustrating functional components of another embodiment of a sample analysis system incorporating elements of a direct sample injection system.

FIG. 3 is a simplified flow diagram illustrating the general operation of one embodiment of a method of performing an analysis using a direct sample injection system.

FIG. 4 is a simplified flow diagram illustrating the general operation of another embodiment of a method of performing an analysis using a direct sample injection system.

FIG. 5 is a simplified diagram illustrating a perspective view of one embodiment of a sample injection guide engaged with a pipette tip during use.

FIG. 6 is a simplified diagram illustrating a perspective view of one embodiment of a coupling component allowing a pipette probe to engage a pipette tip.

FIG. 7 is a simplified diagram illustrating a side elevation view of the coupling component embodiment of FIG. 6.

FIG. 8 is a simplified diagram illustrating an axial view of the coupling component embodiment of FIG. 6.

FIG. 9 is a simplified diagram illustrating a perspective view of one embodiment of a sample injection guide.

FIG. 10 is a simplified diagram illustrating a plan view of the sample injection guide embodiment of FIG. 9.

FIG. 11 is a simplified diagram illustrating a side elevation view of the sample injection guide embodiment of FIG. 9.

FIG. 12 is a simplified diagram illustrating an axial cross-section view of the sample injection guide embodiment of FIG. 9 taken on the line 12-12 in FIG. 10.

FIG. 13 is a simplified perspective diagram illustrating components of one embodiment of a sample analysis system incorporating a direct sample injection system.

FIG. 14 is a simplified perspective diagram illustrating components of one embodiment of a direct sample injection system.

FIG. 15 is a simplified perspective diagram illustrating additional components of the direct sample injection system of FIG. 14.

FIG. 16 is a simplified flow diagram illustrating the general operation of one embodiment of a method of performing an analysis.

FIG. 17 shows a flow diagram of the GOF sorting process, showing inputs, decision points, and outputs (results).

FIG. 18 shows GOF sorting of human serotonin receptor Type 2A populations. Upper figure: Measurements of responses of wild type (WT), variant 3.36 A, and 2.546 A receptors to naphthyl piperizine over a concentration range of from 10⁻⁹M to 10⁻⁴M. Lower figure: Measurements of responses of FACS-sorted cells to naphthyl piperizine over a concentration range of from 10⁻⁹M to 10⁻⁴M.

FIG. 19 shows extended GOF sorting using two test compounds to obtain distinct populations enriched for variant ligand targets uniquely responsive to distinct ligands, L1 and L2. Right panel: GOF receptors for L1 were identified and then screened against L2 and only those L1 GOF receptors that were nonresponders to L2 were selected. Left panel: GOF receptors for L2 were identified and then screened against L1 and only those L2 GOF receptors that were nonresponders to L1 were selected.

FIG. 20 shows extended GOF sorting using two test compounds to obtain distinct populations enriched for variant ligand targets uniquely responsive to distinct ligands, L1 and L2. GOF receptors for L1 were identified and then screened against L2 and only those L1 GOF receptors that were nonresponders to L2 were selected. GOF receptors for L2 were identified and then screened against L1 and only those L2 GOF receptors that were nonresponders to L1 were selected.

### DETAILED DESCRIPTION OF THE INVENTION

The present disclosure provides compositions and methods for high throughput Gain of Function (GOF) sorting to discover and develop novel and highly selective candidate drug molecules. GOF sorting as provided herein identifies those mutations in a ligand target (receptor) and/or ligand that provide increased activity, or gain of function (GOF).

High throughput GOF sorting as provided herein uses rapid functional mutation analysis of large numbers of ligand targets and/or ligands, in combination with high throughput screening and sorting, to identify promising drug candidates. GOF sorting as disclosed herein provides a ‘missing link’ between structure and function, generating powerful structure-activity relationship information regarding key physical and chemical interactions between receptors and ligands, for use in molecular modelling techniques to optimize drug discovery. By generating, identifying, and characterizing a large number of variant ligand targets and/or ligands, the information provided by GOF sorting provides guidance for medicinal chemistry approaches to developing drug candidates having optimal functional activity.

High throughput GOF sorting includes, but is not limited to, mutating a ligand target (receptor) and/or a ligand prior to contacting the ligand target and ligand, and then measuring the functional activity (e.g., affinity and efficacy) of the ligand target-ligand interaction. High throughput GOF sorting further includes carrying out multiple rounds of mutation and measurement, in order to determine which residues in the ligand target and/or the ligand provide key interaction points underlying the functional activity of each ligand target-ligand interaction. As provided herein, the methods are scaleable to, and suitable for, rapidly generating and screening mutations to carry out high throughput analysis. Further, the present disclosure provides methods and compositions for high throughput and high precision GOF sorting, such that large numbers of mutations can be generated and screened rapidly, and GOF ligand target-ligand pairs can be identified and isolated.

In accordance with one aspect, GOF sorting includes mutating single residues of a ligand target (target receptor), generating a plurality of variant ligand targets, contacting the variant ligand targets with a ligand, measuring the functional activity of the ligand target-ligand interaction for each variant ligand target, and determining which residues provide key interaction points on the receptor underlying the functional activity of the ligand. Contacting variant ligand targets with ligands generally occurs by incubating ligand(s) with cells expressing the receptor(s). In certain embodiment, variant ligand targets may be displayed on the surface of synthetic microparticle beads that are also often used for FACS sorting. Techniques for displaying receptor and other proteins on beads for FACS sorting upon ligand binding rely on measuring the binding affinities or kinetics of binding associations; these techniques are known in the field (Simons et al. (2003). Mol Pharmacol 64:1227-38; Sklar et al. (2002). Ann Rev Biophys Biomol Struct 31:97-119.; Young et al. (2004) J Biomol Screen 9(2):103-11.).

In accordance with another aspect, GOF sorting includes mutating a target receptor at multiple sites, preferably within the binding site of the receptor, to determine which residues provide key interaction points underlying the functional activity of the ligand target-ligand interaction. In certain embodiments, single point mutations are generated in G protein coupled receptors (GPCRs) as provided herein. The results generated by GOF sorting expand the information set of the physical and chemical properties of the functional binding site within each receptor studied by GOF sorting.

Although naturally occurring GOF mutations are known for various receptors,. including but not limited to GPCRS, these mutations are not typically described as occurring in the binding pocket, and may include mutations in non-coding regions such as promoters that affect protein expression; Nor is ligand-dependent functional activity typically investigated in naturally occurring GOF mutants. Similarly, although mutational analyses of the binding pocket of various receptors have been performed previously, these analyses have typically been performed one mutation at a time.

It is well known that naturally occurring mutations such as Single Nucleotide Polymorphisms (SNPs) are known to affect the clinical efficacy of drugs, sometimes in proteins other than the drug target that participate in the target mechanism of action, as proven recently for statins. The effort to identify SNPs within the human population relevant to drug efficacy has been referred to as “personalized medicine,” based on the idea that identifying the particular set of SNPs or gene alterations in a given patient may enable a more precise diagnosis and choice of therapeutic treatment. A straightforward application would be when there are several drug treatments available for the patient's indication, and identifying the SNPs of the patient would allow the selection of the most efficacious of the various drug treatments that are available. Current approaches rely on the identification of SNPs in as many patient samples as possible, which is a very costly effort requiring large numbers of patients before statistically significant correlations with drug treatment efficacy can be ascertained. This approach is generally considered adequate to identify the most common SNPs within the population. However, given the statistical occurrence of several SNPs on each gene of each human individual, this approach would, at best, be capable of predicting the effect on drug efficacy for a small subset of the SNPs found in a given patient, i.e., those that are commonly found in other patients. A different approach is provided herein, whereby the potential SNPs or genetic changes in the gene of a drug target (e.g., a receptor) can be artificially generated using molecular biology tools that are known in the art. A “library” of SNPs could be generated, such that library of SNPs for a gene can be expressed and displayed on a cell or other formats such as beads, and the optical isolation process provided herein is applied to identify and isolate those SNPs that have a significant effect on the interaction of the drug with the drug target (receptor), in terms of binding, functional response, expression levels, or a combination thereof. In particular, SNPs showing GOF activity (“GOF SNPs) can be identified and isolated. Optical isolation of GOF SNPs as provided herein would isolate GOF SNPs that enhance that particular drug's activity, and a number of different approaches such as DNA microarrays can be utilized to identify and pharmacologically characterize the isolated GOF SNPs. By iteratively repeating the process, choosing various SNPs as the reference sequence (“control” sequence) rather than the wild type gene, a different set of SNPs can be identified with a different range of effects. For example, one could rank most SNPs into different categories, e.g., SNPS that strongly enhance the effect of the drug; SNPs that partially enhance the effect of the drug; SNPs with neutral effects; SNPs with partially inactivating effects; or SNPs with strongly inactivating effects. This information would be valuable for “personalized medicine” because, for every patient, the SNPs present in genes that are known to be relevant to the drug action can be identified. This information can be submitted to a database containing the measured “in vitro” effects of those SNPs on the activity of the different drug treatments available. This database would identify any SNP known to affect the activity of these available drugs. The physician could then make an “evidence-based” selection of the most appropriate drug for this particular individual based on this data, interpreting the expected clinical phenotype of the pharmacological activity measured in vitro for said SNPs. This approach would identify potential benefits or adverse effects of the SNPs found in the genes of each individual patient, which would be an improvement over current approaches based on the most common SNPs in the general population. The approach described above can also be used to identify SNPs for genes that are associated with (related to or functionally coupled to) the drug target, where these drug-target-associated genes could affect drug efficacy.

In accordance with another aspect, GOF sorting includes mutation of both receptors and ligands. In one embodiment, each variant ligand target is screened against all the variant ligands created for that embodiment. In another embodiment, each variant ligand is screened against all variant ligand targets created for that embodiment. In another embodiment, multiple variant ligand targets are simultaneously screened against multiple variant ligands. GOF sorting as provided herein can be used to sort and identify those receptors and ligands (“GOF ligand target-ligand pairs”) that show enhanced function. These GOF ligand target-ligand pairs are useful in the identification of specific ligand-receptor interactions, based on correlating the GOF effects of a chemical modification in the ligand with a mutation on the receptor, suggesting an interaction between the chemical modification and the residue(s) in the receptor where the GOF mutation occurs.

The methods and compositions provided herein are suitable for high throughput GOF sorting of hundreds or thousands of variant ligand targets. The methods and compositions provided herein permit mutation of any target receptor and generation of any desired quantity of cell populations expressing variant ligand targets. GOF sorting provides methods and compositions to quickly analyze the interactions of hundreds or thousands of ligands and receptors, and identify the few cells expressing GOF variant ligand targets and recover those variants as viable cells for further characterization. It is understood that a small percentage of mutations are expected to give rise to GOF activity. The vast majority of mutations are expected to result in loss of function, no change in function, or a change in processing and/or expression of the receptor.

In accordance with one aspect, high throughput analysis based on pharmacologically defined functional phenotypes is carried out using a suitable fluidics-based sample analysis system. In certain embodiments, high throughput analysis based on pharmacologically defined functional phenotypes is carried out using flow cytometry. In particular, flow cytometry is carried out using a sampling system that can derive pharmacological criteria for hundreds or thousands of samples per day. If desired, cells expressing receptors of interest are isolated and recovered using fluorescence activated cell sorting (FACS). It is understood that GOF sorting as provided herein is not limited to use of flow cytometry and in particular, fluorescence activated cell sorting by flow cytometry, and that other sample handling methods can be adapted for use in accordance with the present disclosure. It is further understood that one of skill in the art can select and adapt a sorting technique for use in the GOF sorting method provided herein, as long as the sorting technique provides the advantages of the present invention. In particular, microfludic devices can be used for fluorescence activated cell sorting, e.g., as described by Kruger et al., (2002), M. Miromech. Microeng. 12:486-494. GOF sorting as provided herein can be carried out using microfluidic optical isolation as described by MacDonald et al., (2003) Nature 426:421-424 or “micro-FACS” as described by Fu et al., (1999) Nature Biotechnology 17:1109. GOF sorting (GOF optical isolation) as provided herein can be carried out by optical fractionation using the “holographic optical tweezers” (HOT) technology described in U.S. Pat. Nos. 6,630,20; 6,626,546; 6,624,940; 6,416,190; and 6,055,106.

Definitions.

“Gain of function” (GOF) as used herein refers to activity that is higher than, or different from, normal activity. Here, “GOF receptor” refers to a variant ligand target that functions with increased efficiency as compared to the wild type receptor. The enhanced function could be either agonism, or antagonism, or inverse agonism, or could also be allosteric modulation (positive or negative), or could also be binding affinities, or could also refer to expression levels of the target receptor, or could be a kinetically different phenotype of any of these properties. Generally, a GOF mutant receptor of the present invention refers to agonism measured by intracellular calcium signals, and said GOF mutant receptor would have a lower EC₅₀ for a given ligand than another receptor construct such as the wild type receptor. In this case, a GOF mutant receptor is activated at a lower concentration of a test compound than the concentration that activates the reference receptor (e.g., a wild type receptor) to produce the same functional response to the test compound. A GOF receptor may be more sensitive due to activity that is enhanced relative to the wild type receptor (hypermorphic GOF), or may have a new activity not found in the wild type receptor (neomorphic GOF). A “GOF ligand target-ligand complex” or “GOF ligand target-ligand interaction” refers to a ligand-receptor interaction that shows a higher level of functional activity, or “gain of function” than the baseline functional activity. A “GOF cell” or “GOF variant cell” refers to a cell expressing a receptor that shows GOF activity with respect to a test compound. It is understood that “GOF” is a relative term, and refers to gains in sensitivity or efficacy with respect to a particular test compound, or a particular receptor.

The present disclosure provides systems that are useful for measuring the interaction between a receptor and a ligand. In particular, the present disclosure provides systems that are useful for measuring the interaction between a drug target and a drug candidate. “Receptor” or “receptor target” as used herein is intended to mean any ligand-binding protein including, but not limited to, ligand-binding G-protein-coupled receptors (GPCRs), ligand-gated ion channels, ligand-binding proteins having a single transmembrane domain, ligand-binding proteins that translocate across a cell membrane after binding, ligand-binding enzymes, ligand-binding regulators of gene expression, ligand-binding structural proteins, or any other protein wherein ligand binding triggers a cellular response. “Receptor” or “receptor target” are used interchangeably with “ligand target.,” and are intended to mean any ligand-binding molecule, including but not limited to, RNA, DNA, PNA, LNA, aptamers, or any viable biological target. Receptor targets to be screened may be wild type receptors or mutated variant ligand targets. In accordance with various aspects of the invention, these targets may be endogenously expressed receptors, or may be receptors expressed as a result of the introduction of exogenous sequences expressed by means of regulatable or non-regulatable transfection and expression.

“Wild type receptor” refers to an existing, naturally occurring version of a receptor. “Variant ligand target” can refer to a naturally occurring variant, e.g., a SNP, an allele or a splice variant of the gene encoding the wild type drug target, or to a variant created by deliberate modification or mutation of the DNA encoding the receptor. The term “DNA encoding a receptor” encompasses both the DNA sequence that is transcribed and translated to produce the receptor (i.e., the coding region), and associated regulatory regions that may or may not be transcribed or translated.

“Ligand” is used to mean any compound being tested in the system disclosed herein, where it is understood that the term generally refers to ligands that bind to the receptors being screened. Ligand can thus be defined as a molecule that is added to a mixed population of variant ligand targets, followed by optical isolation of those variant ligand targets having a GOF phenotype towards the molecule. Ligands of the present invention may be natural ligands, modified ligands, mutated ligands, synthetic compounds that are not naturally occurring, synthesized versions of naturally occurring ligands, or any test compound(s) suitable for use as a test compound in the screening system provided herein. The term ligand includes, but is not limited to, chemical compounds and biological molecules such as peptides, proteins, oligonucleotides, oligonucleosides, RNA, DNA, PNA, or LNA. Ligands can also be antigens or epitopes screened against mutant antibody targets functioning as receptors. Conversely, ligands can be antibodies screened against mutant antigens or epitopes functioning as receptors. Similarly, ligands can be substrates or their corresponding enzymes, depending on whether a given substrate molecule (ligand) is screened against mutants of the enzyme (receptor), or whether a given enzyme (ligand) is screened against mutants of the substrate (receptor). This definition includes the screening of a library of ligands, one at the time, against a set of mutant receptors, to identify GOF mutant receptors for different ligands. It is further understood that the term “ligand” may be used to refer to a compound that interacts with a ligand-receptor complex, in such a way as to modulate the functional activity of the ligand-receptor complex. The term “drug candidate” refers to a ligand that has been identified as having properties that have potential therapeutic use. “Wild type ligand” refers to an existing, naturally occurring version of a receptor. “Variant ligand” can refer to a naturally occurring variant of a ligand, or to a variant ligand created by deliberate modification of the ligand or mutation of the DNA encoding the ligand. The term “DNA encoding a ligand” encompasses both DNA sequence that is transcribed and translated to produce the ligand (i.e., the coding region), and associated regulatory regions that may or may not be transcribed or translated.

The terms “functional activity” and “functional response” refer to cellular responses measured in a cell, triggered by the interaction between a ligand and ligand target (receptor) on the cell. “Functional activity” or “functional response” encompass both the affinity and efficacy of a ligand target-ligand interaction, where “affinity” refers to the concentration of ligand or ligand target at which a desired level of cellular response is measured, and “efficacy” refers to the level of cellular response triggered by the interaction of a ligand and a ligand target. It is understood that affinity is generally determined by varying the concentration of ligand and keeping the amount of ligand target constant in each assay of a GOF sorting experiment, but affinity can also be determined by varying the amount of ligand target present if desired. Functional responses (cellular responses to a ligand-receptor interaction) include, but are not limited to, changes in receptor structure or function, ion fluxes across cell membranes, in particular ion fluxes causing changes in Ca²⁺ᵢ levels, changes in cell size or shape, cell proliferation, cell differentiation, modulation of the activity of enzymes coupled to the drug target, alteration in genetic expression, and cell death. The present disclosure provides methods for measuring functional responses that are suitable for use in the screening systems of the present invention. In particular, the present disclosure provides functional target-coupled readout systems for measuring functional responses. The terms “functional activity” and “functional response” likewise refer to responses or effects measured on beads displaying the ligand or the receptor, where the response is triggered by the interaction between the ligand and receptor, wherein “beads” refers to particles used to display ligands or receptors in a format suitable for optical isolation of GOF mutant receptors. It is understood that, for a particular embodiment, one of skill in the art can select a cellular response appropriate for assessing the functional activity of the ligand-receptor interaction of the embodiment, and further that one of skill in the art can select a functional target-coupled readout system for measuring that functional response.

It is understood that the choice of terms such as “ligand”” and “drug candidate” and “receptor” are not intended to limit the scope of ligands or ligand-binding proteins that are suitable for use in the GOF sorting systems provided herein. It is further understood that the choice of the term “functional response” is not intended to limit the scope of biological responses that are suitable for use in the GOF sorting systems provided herein.

“Flow cytometer,” “flow cytometry,” and “fluorescence activated cell sorting (FACS)” refer to well-known methods and tools described in numerous U.S. patents and scientific references, inter alia, U.S. Pat. No. 3,826,364; U.S. Pat. No. 3,826,412; U.S. Pat. No. 4,600,302; U.S. Pat. No. 4,660,971; U.S. Pat. No. 4,661,913, U.S. Pat. No. 4,988,619; U.S. Pat. No. 5,092,184; US U.S. Pat. No. 5,994,089; U.S. Pat. No. 5,968,738; U.S. Pat. No. 6,014,904; U.S. Pat. No. 6,248,590; U.S. Pat. No. 6,256,096; U.S. Pat. No. 6,664,110; U.S. Pat. No. 6,680,367; Haynes, “Principles of Flow Cytometry” (1988) Cytometry Supplement 3:7-18; Ormerod (ed.), Flow Cytometry: A Practical Approach, Oxford Univ. Press (1997); Jaroszeski et al. (eds.), Flow Cytometry Protocols, Methods in Molecular Biology No. 91, Humana Press (1997); and Shapiro, Practical Flow Cytometry, 3rd Ed., Wiley-Liss (1995). The term “FACS” as used herein, refers both to a fluorescence-activated cell sorting apparatus, i.e., an instrument based on flow cytometry that can select one cell from thousands of other cells, and to the method of fluorescence-activated cell sorting. All remaining terms have their usual meaning in the flow cytometric arts, as set forth, inter alia, in Haynes, “Principles of Flow Cytometry” (1988) Cytometry Supplement 3:7-18; Ormerod (ed.), Flow Cytometry: A Practical Approach, Oxford Univ. Press (1997); Jaroszeski et al. (eds.), Flow Cytometry Protocols, Methods in Molecular Biology No. 91, Humana Press (1997); and Shapiro, Practical Flow Cytometry, 3rd Ed., Wiley-Liss (1995).

GOF Sorting Using Variant Ligand Targets

Creating Variant Targets.

In accordance with one aspect of the present invention, a first step involves developing one or more cell populations bearing receptors, where the cell populations bearing receptors are to be screened for their interactions with ligands. In accordance with one aspect, cellular responses of cell populations bearing ligand targets (receptors) are measured, in order to measure interactions between ligand targets and ligands. Measuring functional responses triggered by interactions between ligands and multiple ligand targets provides a multiplexed screening method that allows identification of those ligands and receptors that interact, and further allows measurements of the affinity and efficacy (amount of cellular response) of the interaction. In certain embodiments, cells bearing wild type ligand targets (wild type receptors) are screened for their interactions with ligands. In certain embodiments, cells bearing variant or mutant ligand targets (variant or mutant recptors) are screened for their interactions with ligands.

“Wild type” refers to an existing version of the receptor. “Variant” is used to refer to a ligand target (receptor) that differs from the one that has been identified as the wild type for that ligand target (receptor). As used herein, “variant” can refer to a naturally occurring variant, e.g., a SNP, an allele or a splice variant of the gene encoding the wild type receptor, or to a variant created by deliberate modification or mutation of the DNA encoding the receptor, or to a chemical modification of the resulting receptor protein. “Mutant” refers to a receptor encoded by DNA that has been deliberately modified or mutated to encode a mutant receptor that differs from the wild type receptor. The term “variant” may be used alone, or the terms “variant” and “mutant” may be used in combination in the present disclosure, to encompass all receptors that differ from the wild type receptor. The term “DNA encoding a receptor” is intended to encompass a scope similar to “receptor gene” referring both to DNA sequence that is transcribed and translated into the receptor protein (the coding region) and to associated regulatory regions that may or may not be transcribed or translated.

Variant or mutant receptors are most commonly generated using standard molecular biology techniques to mutate or otherwise modify DNA encoding the receptor, where the DNA is modified at one or more residues. Mutations or modifications of DNA encoding the receptor include, without limitation, additions, deletions, substitutions, duplications, and rearrangements, further including engineered splice variants. DNA encoding a receptor can be complementary DNA (cDNA) reverse-transcribed from messenger RNA (mRNA). In accordance with the present invention, both coding and non-coding (regulatory) regions of DNA can be modified or mutated to produce a mutant receptor. Standard techniques for modification or mutation of DNA to generate mutated receptors include “shotgun” mutagenesis, cassette mutagenesis, chemical mutagenesis, site-directed mutagenesis, in situ mutagenesis, “directed evolution” (e.g., as described in U.S. Pat. No. 6,531,580), mutator strain induced mutagenesis, RNA-DNA chimeroplasty for targeted mutagenesis, DNA shuffling, error-prone PCR, other combinatorial techniques, or other standard techniques as found, for example, in Sambrook et al., Molecular Cloning, A Laboratory Manual, (3^{rd} Ed. (2000), 2^{nd} Ed. (1989), Cold Spring Harbor Laboratory Press, N.Y., or Ausubel et al., Eds. Current Protocols in Molecular Biology, (1991 and updates) Wiley Interscience, N.Y.

Receptors may be expressed from DNA already present in a cell, or may be expressed from DNA that has been introduced into a cell. Wild type, variant, and mutant receptors may be expressed from DNA already present in a cell. In one non-limiting example, a mutant receptor is expressed from mutated DNA already present in a cell, where the cell has been subjected to chemical mutagenesis using EMS. In accordance with this aspect, the expression of receptors is regulated by endogenous regulatory elements such as promoters, enhancers, activators, or repressors. In one embodiment, receptors expressed from DNA already present in a cell are constitutively expressed under the control of constitutive promoters or enhancers. In another embodiment, expression of receptors expressed from DNA already present in a cell is under the control of inducible regulatory elements, e.g., inducible promoters or repressors, and expression depends on manipulation of these regulatory elements.

Wild type, variant, and mutant receptors may be expressed from DNA that has been introduced into a host cell. The term “transfection” is intended to include any means by which a nucleic acid molecule can be introduced into eukaryotic or prokaryotic cells. The introduced nucleic acid molecule can be DNA or RNA, and may be either single or double-stranded; in the present disclosure, the introduced nucleic acid molecule is referred to as DNA. As used herein, “transfection” encompasses both transient cell transfection, wherein the DNA encoding a receptor is transiently expressed, and stable transformation of cells, wherein the DNA encoding a receptor is maintained by integration into chromosomal DNA or persistence in a stable extrachromosomal element. DNA used in transfection of host cells can be circularized, e.g., in a vector (plasmid) or may be linear, depending on the transfection and expression method selected for a particular embodiment. “Recombinant” expression of receptors refers to transfection of host cells and expression of the introduced DNA encoding receptors.

In accordance with one aspect, a host cell is transfected with DNA encoding a wild type receptor, producing a cell bearing a recombinantly expressed wild type receptor. In accordance with another aspect, a host cell is transfected with DNA encoding a variant or mutant receptor, producing a cell bearing a recombinantly expressed variant or mutant receptor.

Suitable transfection methods include, but are not limited to, a variety of techniques useful for introduction of nucleic acids into mammalian cells including electroporation, calcium phosphate precipitation, DEAE-dextran treatment, lipofection, microinjection, and viral infection. Suitable methods for transfecting mammalian cells can be found in Sambrook et al., Molecular Cloning, A Laboratory Manual, (3^{rd} Ed. (2000), 2^{nd} Ed. (1989), Cold Spring Harbor Laboratory Press, N.Y., or Ausubel et al., Eds. Current Protocols in Molecular Biology, (1991 and updates) Wiley Interscience, N.Y. and other laboratory textbooks. In accordance with one aspect, non-viral-mediated methods for introducing DNA into a host cell include use of a cell-delivery vehicle such as cationic liposomes or derivatized (e.g., antibody conjugated) polylysine conjugates, gramicidin S, or artificial viral envelopes, e.g. as described in Philip et al,. (1994) Mol Cell Biol 14:2411. In another embodiment, DNA encoding a receptor is delivered into a host cell in the form of a soluble molecular complex including a nucleic acid binding agent and a cell-specific binding agent, such that the complex binds to the host cell surface and is subsequently internalized by the cell, e.g., as described in U.S. Pat. No. 5,166,320. In another embodiment, DNA encoding a receptor is introduced into a host cell by particle bombardment, as described in Yang and Sun (1995) Nature Medicine 1:481. In accordance with another aspect, DNA encoding receptors can be introduced using vectors, e.g., viral vectors including but not limited to recombinant retroviruses, adenovirus, adeno-associated virus, and herpes simplex virus-1.

One suitable transfection method is known as Single Target Integration Site (STIS), wherein a single DNA copy of the sequence encoding a receptor is transfected into a population of target cells that lack the target receptor but that bear a single target integration site (STIS), and the DNA is stably integrated into the genome of each target cell. Using STIS, only one DNA sequence is successfully transfected into, and expressed by, each host cell, and each DNA is integrated into the identical genomic locus in each cell. This can be accomplished using standard homologous recombination techniques known in the art such as the Cre/Lox or Flip-in systems. The required Lox or Flip-in DNA segments are stably integrated first in the host cell genome, flanked by DNA sequences recognized by specific recombinase enzymes. The DNA encoding the receptor is then co-transfected together with the DNA encoding the recombinase, so that the recombinase enzyme can facilitate integration of the receptor encoding DNA into the pre-established lox or Flip-in DNA segments. This method enables the transfection of a mixed population of variant ligand targets (a variant ligand target library) into a host cell line, resulting in a cell population stably expressing some of these variant ligand targets, whereby each individual cell would typically expresses only one single variant ligand target. A single variant ligand target per cell ratio facilitates the optical isolation such as FACS sorting of GOF mutant receptors because these systems function by isolating individual cells, which in this case efficiently isolate individual mutant receptors. Because the different variant ligand targets in different cells would be integrated in the same locus within the genome of the same host cell, this technique minimizes cell-to-cell variation for each variant such as expression levels and expression cycles. This technique thus facilitates the interpretation of the phenotype of different cells expressing different variant ligand targets based on the effect of the variant ligand target. This is important because optical isolation such as FACS sorting of GOF variant ligand targets relies on a GOF phenotype measured in the individual cells but whose interpretation is based on the specific mutation of the variant ligand target. In one embodiment, STIS is selected to maximize homogeneity of assay results.

Another suitable transfection method is Random Target-Integration Site (RTIS). A standard transfection technique such as lipofectamine or electroporation, is used to transfect a cell population. It should be noted that, since there is no assurance that each cell integrates the transfected DNA at the same location in the genome, RTIS can lead to different expression levels between cells within the population. Different expression levels normally affect the function of the receptor, and thus random integration could potential lead to more false positives where the detection and isolation of GOF phenotypes on individual cells may be due to different expression levels of the mutant receptor rather than to specific ligand-receptor interactions. However, such cases can be identified by measuring the dependence of the cellular activity on the expression levels of the mutant receptors, as it is commonly practiced in the art.

In certain embodiments, the transfection step can be performed such that an entire library of mutated DNAs encoding receptors is transfected simultaneously. In these cases, when applied to mammalian host cells, the STIS approach would yield a one variant per cell ratio in the resulting mixed variant population, but RTIS may result in multiple variant ligand targets expressed in the same cell. If multiple variants are expressed per cell, optical isolation such as FACS sorting would isolate GOF cells but it would be more challenging to identify the specific variant ligand target responsible for the observed GOF phenotype, requiring a deconvolution step. Alternately, an entire library of mutated DNAs encoding receptors is transfected individually in separate cell pools that are combined later. This step insures that each cell constitutes a single variant ligand target-bearing assay system (variants). In certain embodiments, transfection of the wild type (WT) receptor using the same single target integration site (STIS) technology is included as a control for comparison with the variant ligand target population responses.

It is understood that expression of receptors may be affected by designing the DNA molecule to be introduced into host cell, to include various sequences that can regulate expression of DNA encoding a receptor. Such a molecule typically contains regulatory elements to which the DNA encoding a receptor is operably linked in a manner which may influence transcriptional, translational, or post-translational events related to expression of the receptor in host cells. Regulatory elements are selected to direct expression of the receptor in a suitable host cell and include, but are not limited to, promoters, enhancers, polyadenylation signals, and sequences necessary for transport of the receptor to the appropriate cellular compartment (usually, insertion into the cell membrane). When the introduced DNA is a cDNA in a recombinant expression vector, the regulatory element controlling transcription and/or translation of the cDNA are often derived from viral sequences. Regulatory elements are known in the art and are described in, e.g., Goeddel, Gene Expression Technology: Methods in Enzymology 185, Academic Press, San Diego, Calif. (1990).

Regulatory sequences linked to the DNA encoding the receptor include promoters that can be selected to provide constitutive or inducible transcription. Suitable promoters for use in various systems are known in the art. For example, suitable promoters for use in murine cells include RSV LTR, MPSV LTR, SV40 IEP, and metallothionein promoter, and CMV IEP is a suitable promoter for use in human cells. Examples of commonly used viral promoters include those derived from polyoma, Adenovirus 2, cytomegalovirus and Simian Virus 40, and retroviral LTRs.

In a specific embodiment, DNA encoding the receptor is under the control of an inducible control element such as an “inducible promoter” or enhancer, such that expression can be regulated by contacting (or not contacting) the host cell with an agent which affects the inducible control element. Inducible regulatory systems for use in mammalian cells are known in the art, for example systems in which gene expression is regulated by heavy metal ions (Mayo et al., (1982) Cell 29:99-108; Brinster et al., (1982) Nature 296:39-42; Searle et al., (1985) Mol Cell Biol 5:1480-1489), heat shock (Nouer et al. (1991) in Heat Shock Response, Nouer, ed., CRC, Boca Raton, Fla., pp167-220), hormones (Lee et al. (1981) Nature 294:228-232; Hynes et al. (1981) Proc. Natl. Acad. Sci. USA 78:2038-2042; Klock et al. (1987) Nature 329:734-736; Israel & Kaufinan (1989) Nuc. Acids Res. 11:2589-2604 and PCT Publication No. WO 93/23431), tetracycline (Gossen, M. and Bujard, H. (1992) Proc. Natl. Acad. Sci. USA 89:5547-5551 and PCT Publication No. WO 94/29442) or FK506 related molecules (PCT Publication No. WO94/18317).

In another embodiment of the invention, DNA encoding a receptor is under the control of regulatory sequences such as “constitutive promoters” or constitutive enhancers which constitutively drive the expression of the DNA encoding a receptor Exemplary constitutive promoters include, but are not limited to, the promoters for the following genes: hypoxanthine phosphoribosyl transferase (HPRT), dihydrofolate reductase (DHFR) (Scharfmann et al., Proc. Natl. Acad. Sci. USA 88: 4626-4630 (1991)), adenosine deaminase, phosphoglycerol kinase (PGK), pyruvate kinase, phosphoglycerol mutase, the β-actin promoter (Lai et al., Proc. Natl. Acad. Sci. USA 86: 10006-10010 (1989)), and other constitutive promoters known to those of skill in the art. In addition, many viral promoters function constitutively in eucaryotic cells including, but not limited to, early and late promoters of SV40 (See Bemoist and Chambon, Nature, 290:304 (1981)); long terminal repeats (LTRs) of Moloney Leukemia Virus and other retroviruses (See Weiss et al., RNA Tumor Viruses, Cold Spring Harbor Laboratory, Cold Spring Harbor, N.Y. (1985)); thymidine kinase (TK) promoter of Herpes Simplex Virus (HSV) (See Wagner et al., Proc. Nat. Acad. Sci. USA, 78: 1441(1981)); cytomegalovirus immediate-early (IE1) promoter (See Karasuyama et al., J. Exp. Med., 169: 13 (1989); Rous sarcoma virus (RSV) promoter (Yamamoto et al., Cell, 22:787 (1980)); adenovirus major late promoter (Yamada et al., Proc. Nat. Acad. Sci. USA, 82: 3567 (1985)), and other viral-derived constitutive promoters known to those of skill in the art

Host Cells

It is understood that suitable host cells may be chosen according to the characteristics of each embodiment. In accordance with one aspect, suitable host cells for transfection with DNA encoding a receptor are cells that do not normally express the receptor. In these cells, the cellular responses that are measured are considered to reflect the interaction of the receptor with the ligand. Generally, mammalian host cells are suitable for GOF sorting. Other host cells, especially eukaryotic cells, may be suitable for certain embodiments.

In accordance with another aspect, host cells that normally express the wild type receptor are transfected with DNA encoding a variant or mutant receptor. In host cell that normally express the receptor, the baseline cellular responses characteristic of the normal cell expressing a wild-type receptor are known, such that differences from that baseline may be ascribed to the effect of also expressing a variant or mutant receptor. It is understood that choice of host cell may be determined by choice of expression vector. Examples of suitable host cells include, but are not limited to, HEK293, U937, COS-7, NIH/3T3, HeLa, and CHO cell lines.

In certain embodiments, non-mammalian host cells may be suitable, including but not limited to, Drosophila melanogaster S2 cells, Spodoptera frugiperda Sf9 cells, High-5 cells, yeast cells including Saccharomyces species or Pichia species, and bacterial cells e.g., E. coli. Yeast cells and bacterial cells have the advantageous property that transfection of a mixture of vectors encoding different variant ligand target constructs results in most cells expressing one single variant ligand target per cell, due to their smaller size.

The suitability of a particular cell for use as a host cell in accordance with the invention will depend on the ability to introduce a DNA encoding a receptor into the cell, and express the receptor. Cells may be adherent or non-adherent. Host cells may be chosen or developed on the basis of certain desirable properties of the precursor cells of the host cells. It is understood that one of skill in the art will select a suitable host cell according to goals, characteristics, conditions, and/or constraints of a particular embodiment.

In accordance with another aspect, host cells that already express a receptor may be treated to induce expression of a variant or mutant receptor without transfection with DNA encoding a receptor. One suitable method involves in situ mutagenesis of a host cell expressing a receptor, e.g., by chemical or radiation mutagenesis. Another suitable method involves insertional mutagenesis, e.g., transposon mutagenesis, of a host cell expressing a receptor. In one embodiment, the host cell already contains one or more transposons that are activated. In another embodiment, one or more transposons are introduced into the host cell. After mutagenesis, cells are screened to identify those cells expressing variant or mutant receptors.

Screening Variants.

Cells bearing variant and WT receptors are screened as provided herein. In accordance with one aspect of the invention, one or more populations of cells bearing variant ligand targets are developed for use in a particular embodiment. Generally, at least one population of cells bearing the corresponding wild type (WT) receptor is developed to provide control, or baseline, values for each embodiment. The control untransfected host cell is added to identify false positives GOF phenotypes arising from cellular responses unrelated to the intended receptor target and variant constructs. As provided herein, cells from a plurality of cell population are combined to form a mixed cell suspension, the mixed cell suspension is exposed to a test compound, a sample containing the mixed cell suspension and the test compound is introduced into the sample analysis system and analyzed. Cells in suspension are required for flow cytometry and microfluidic instrumentation and therefore, the present disclosure focuses on suspension cells to describe the embodiments. However, equivalent embodiments can be conceived for other optical isolation instrumentation such as laser microdissection or laser ablation on microscopes that require adherent cells.

In accordance with one aspect of the invention, the mixed cell suspension is contacted with test compounds and incubated for defined periods of time, after which the mixed cell suspension is rapidly analyzed at a single cell level to determine the functional response of each cell in the suspension. It is understood that the cells have been labelled with a functional target-coupled readout system to measure the functional response. GOF sorting as provided herein utilizes high speed single cell analysis, in contrast to certain commonly used screening methods that measure functional responses to a test compound by determining the average response of a population of cells. In certain embodiments, GOF sorting is carried out in a sequential manner, such that each mixed cell suspension is contacted with one test compound and is introduced into the sample analysis system as a discrete sample. In particular, the discrete samples (mixed cell suspension and test compound) are introduced into the sample analysis system as rapidly as possible in an automated manner. It is understood that GOF sorting provides methods and apparatus whereby these samples are introduced both rapidly and discretely, so there is no (or minimal) intermingling of the discrete samples. GOF sorting further provides single cell analysis and data reduction techniques that distinguish GOF sorting from other screening systems. In certain embodiment, the multiparametric properties of the flow cytometer are utilized so that the functional responses of many different populations of cells can be analyzed simultaneously or within a very short period of time, in contrast to other systems where samples are input as a continuous stream of successive plugs and only one or a few parameters are measured for each sample.

In accordance with another aspect, cells showing a desired response are optionally isolated from the mixed cell suspension and sorted into separate vessels for further characterization (see Step 3 below). Cells could be isolated during the initial screening of a mixed cell suspension, or during a subsequent screening run wherein compounds that were determined to be “hits” in a previous screening are used to isolate responding cells. To carry out both steps, the analysis can be performed using flow cytometry and fluorescence activated cell sorting (FACS) instrumentation. It is understood that the initial component of sorting without screening would be an intrinsic part of the GOF sorting method provided herein.

In accordance with one aspect of the invention, cells and drug candidate are mixed and injected using an automated sample mixing and injection method and apparatus as described in detail in the following section entitled “Direct mixing and injection for high throughput fluidic systems” and in the co-pending patent applications entitled “Direct sample mixing and injection for high throughput fluidic systems” and “Sample analysis system employing direct sample mixing and injection” filed May 7, 2004, the entire contents of which are hereby incorporated by reference.

Identifying Ligand-GOF Ligand Target Combinations; Functional Target-Coupled Readout System to Measure Functional Responses.

GOF sorting as provided herein identifies variant ligand targets that show functional responses towards ligands at concentrations, kinetics, or signal intensities outside the usual range, in particular GOF responses. These different sorting parameters can be utilized alone or in combination for sorting decisions, and would be selected based on the desired phenotypes by one skilled in the art. A process for sorting at lower concentrations is described herein, and it is understood that a similar process can be used for sorting other parameters and combination of parameters. Although ligands may sometimes trigger functional responses in cells bearing the variant ligand targets when the assay is performed at normal screening concentrations, that result may not necessarily be due to a gain of function (GOF) variant. In accordance with the present invention, positive identification of GOF activity is generally achieved by either: (1) screening at atypically low concentrations of compounds to determine which compounds activate the variant but not WT receptor; or 2) performing full dose-response curve analyses with the compound against the variant ligand target(s) and the WT receptor, and comparing the EC₅₀ and Eₘₐₓ (maximal efficacy) values of the compound for the variant and WT receptor populations. In one embodiment, compounds that show increased Eₘₐₓ values or higher efficacies (reduced EC₅₀ values) against some of the variant ligand targets in a first screening are used in a second screening of mixed cell suspension containing naive variant ligand target populations, at a single concentration lower than the EC₅₀ value determined in the previous screen (the “characterization” step or screen). Only those cells having GOF receptors are expected to show a functional response to the low concentration of test compound, and those cells can then be “recognized” during analysis and sorted, e.g. using a flow cytometer in sorting mode. The concentration threshold to isolate GOF variant ligand targets is thus relative to the reference receptor construct, which normally would be the wild type receptor but could also be any variant ligand target.

In accordance with another aspect of the present invention, cells of the present invention are labelled with one or more functional target-coupled readout systems to measure functional responses. Generally, cells are labelled with functional target-coupled readout systems by loading with one or more “indicator dyes” to monitor functional responses triggered by the interaction of a ligand target with a ligand. As provided herein, functional responses measured by functional target-coupled readout systems are changes in cellular physiology including, but not limited to, mobilization of internal Ca²⁺ (Ca²⁺ᵢ) stores, changes in membrane potential, changes in cytoplasmic or intraorganellar pH, and changes in intracellular concentrations of various ions other than Ca²⁺. “Indicator dyes” suitable for use as functional target-coupled readout systems include nucleic acid stains that indicate relevant cellular reponses, e.g., Hoechst 33342 can be used as a viable DNA stain to monitor a functional response that may include chromatin fragmentation and/or apoptosis. The present disclosure provides methods for measuring functional responses can be measured in entire cells or in organelles, e.g., ion concentrations can be measured in the cytoplasm or in organelles such as mitochondria, nuclei, chloroplasts, endoplastic reticulum (ER), Golgi apparatus, or proteasomes, and membrane potential can be measured across the plasma membrane and/or across organellar membranes.

In accordance with one aspect, intracellular Ca²⁺ (Ca²⁺ᵢ) is monitored as an indicator of mobilization of internal Ca²⁺ stores in response to the interaction of a receptor with a ligand. In certain embodiments, the functional target-coupled readout system for measuring Ca²⁺ᵢ in cells and organelles (e.g., mitochondria) includes fluorescent Ca²⁺ indicators as described in The Handbook of Fluorescent Probes and Indicators, 9^{th} Ed., Chapter 20, (Molecular Probes, InVitrogen; available online as “The Handbook, Web Edition” at http://www.probes.com/handbook) Suitable Ca²⁺ᵢ indicator dyes include, but are not limited to, Indo, Fluo, BAPTA indicators available from InVitrogen (Carlsbad Calif.), in particular Indo-1, Fluo-3, Fluo-4, Oregon Green 488 BAPTA, Calcium Green, X-rhod-1 and Fura Red indicators and their variants, which allow Ca²⁺ᵢ detection over a wide concentration range. In certain embodiments, fluorescent indicators are conjugated to high- or low-molecular weight dextrans for improved cellular retention and less compartmentalization. In certain embodiments, fluroescent indicators can be conjugated to lipophilic Ca²⁺ indicators for measuring near-membrane Ca²⁺. Suitable methods for loading cells with Ca²⁺ᵢ indicators include using the acetoxy methyl ester (AM) form of the dye at a concentration of from 0.5 to 10 μM, from a stock solution in DMSO, to the cell suspension while the cells are being stained with color-coding dyes. Generally, the total DMSO is kept at about 1% or less by volume in the cell suspension. Generally, cells are wrapped in foil to prevent photobleaching of any of the probes by ambient light. Cells are generally placed on a rotating or rocking platform to keep the cells in suspension. The cells are incubated in suspension at a suitable temperature for a suitable amount of time, often between about 30 to about 90 minutes, typically about 60 minutes. Depending on the particular embodiment, cells may be incubated at room temperature, e.g., 25° C., or at lower or higher temperatures, e.g., 37° C. One of skill in the art can determine suitable labelling and incubation conditions for each particular embodiment.

In accordance with another aspect, the concentrations of other divalent cations are monitored in response to the interaction of a ligand target with a ligand. In certain embodiments, divalent cations including, but not limited to, Mg²⁺, Zn²⁺, Ba²⁺, Cd²⁺, and Sr²⁺, in cells and organelles (e.g., mitochondria) is measured using a functional target-coupled readout system that includes fluorescent indicators as described in The Handbook of Fluorescent Probes and Indicators, 9^{th} Ed., Chapter 20, (Molecular Probes, InVitrogen; available online as “The Handbook, Web Edition” at http://www.probes.com/handbook). In accordance with one aspect, zinc concentrations can be measured using fluorescent indicators nominally designed for Ca²⁺ detection such as fura-2, or using fluroescent indicators with greater Zn2+selectivity, such as FuraZin-1, IndoZin-1, FluoZin-1, FluoZin-2, and RhodZin-1 (all available from InVitrogen), which detect Zn²⁺ in the 0.1-100 μM range with minimal interfering Ca²⁺ sensitivity, or using Zn²⁺ indicators that have essentially no sensitivity to Ca²⁺, e.g., Newport Green DCF and Newport Green PDX (available from InVitrogen). The spectral responses of these indicators closely mimic those of the similarly named Ca²⁺ indicators, e.g., FuraZin-1 and IndoZin-1 exhibit Zn²⁺-dependent excitation and emission spectral shifts, respectively, and FluoZin-2 and RhodZin-1 show Zn²⁺-dependent fluorescence without accompanying spectral shifts (see, Handbook of Molecular Probes, supra, Chapter 20).

When indicator dyes with similar spectral responses are selected, it is understood that cell populations may be further differentiated for proper identification and sorting of each cell. In one embodiment, each cell population that is added to the mixed cell suspension has been “color-coded” by staining with one or more fluorochromes to yield a distinct optimal signature for cells from that cell population, as described in the patent application entitled “Multiplexed Multitarget Screening Method” filed May 7, 2004, the entire contents of which are hereby incorporated by reference. In one embodiment, “color-coding” or otherwise marking cells with a distinct optical signature allows GOF sorting of cells containing the above-mentioned zinc indicators to be analyzed in a mixed cell suspension containing, e.g., Ca²⁺ indicators having similar spectral responses, since the spectral response of each cell will be correlated with the distinct optical signature of that cell.

In accordance with another aspect, membrane potential is monitored as an indicator of transmembrane ion fluxes in response to the interaction of a receptor with a ligand, where the ions carry sufficient charge to change the electrochemical potential across a membrane. In certain embodiments, membrane potential in cells and organelles (e.g., mitochondria) is measured using functional target-coupled readout systems that include potentiometric optical probes as described in The Handbook of Fluorescent Probes and Indicators, 9^{th} Ed., Chapter 23, (Molecular Probes, InVitrogen; available online as “The Handbook, Web Edition” at http://www.probes.com/handbook) and in Celis, Ed., Cell Biology: A Laboratory Handbook, 2nd Ed., Vol. 3, pp. 375-379 (1998) In accordance with this aspect, potentiometric optical probes are used to detect changes in membrane potential in response to the interaction of a receptor with a ligand. Increases and decreases in membrane potential, or membrane hyperpolarization and depolarization, respectively, play a central role in functional responses involved in, e.g., nerve-impulse propagation, muscle contraction, cell signaling and ion-channel gating. Potentiometric probes are important tools for studying these cellular processes, and for assessing cell viability, for high-throughput screening for new drug candidates. Potentiometric probes include, but are not limited to, the cationic or zwitterionic styryl dyes, the cationic carbocyanines and rhodamines, the anionic oxonols and hybrid oxonols, merocyanine 540, and JC-1. It is understood that one of skill in the art can select the dye for use in a particular embodiment, based on factors such as accumulation in cells, response mechanism and toxicity. In conjunction with imaging techniques provided herein, these probes can be employed to map variations in membrane potential across excitable cells with high levels of sampling frequency and spatial resolution.

In accordance with yet another aspect, the intracellular concentration of any one of various other cations, e.g., Na⁺ or K⁺ or anions, e.g., Cl⁻, phosphate, pyrophosphate, nitrate, or sulfate, as an indicator of functional responses to the interaction of a receptor with a ligand. In certain embodiments, these ion concentrations are measured using indicators as described in The Handbook of Fluorescent Probes and Indicators, 9^{th} Ed., Chapter 22, (Molecular Probes, InVitrogen; available online as “The Handbook, Web Edition” at http:)://www.probes.com/handbook). Suitable cation indicators include, but are not limited to, benzofuranyl fluorophores linked to a crown ether chelator, e.g., PBFI and SBFI available from InVitrogen (Carlsbad Calif.), where cation selectivity is conferred by the cavity size of the crown ether. In certain embodiments, when a cation binds to SBFI or PBFI, the indicator's fluorescence quantum yield increases, its excitation peak narrows and its excitation maximum shifts to shorter wavelengths, causing a significant change in the ratio of fluorescence intensities excited at 340/380 nm. Suitable chloride (Cl⁻) indicators include, but are not limited to, 6-methoxy-N-(3-sulfopropyl)quinolinium (SPQ), N-(ethoxycarbonylmethyl)-6-methoxyquinolinium bromide (MQAE), 6-methoxy-N-ethylquinolinium iodide (MEQ) or lucigenin, all available from InVitrogen (Carlsbad Calif.). Monochlorobimane is a fluorochrome that can be used as a glutathione probe.

Although FACS sorting is hereby described based on fluorescence signals, the parameters that available instrunentation can measure for optical isolation of GOF variant ligand targets include, but are not limited to, morphology, fluorescence, fluorescence polarization, fluorescence lifetime, incident light scatter, electromagnetic field induction, light absorbance, luminescence, fluorescence resonance energy transfer (FRET), and bioluminescent resonance energy transfer BRET).

Cell Sorting.

In accordance with one aspect of the invention, a test compound can be screened against cell populations bearing WT and variant ligand targets, and cells bearing GOF ligand targets with respect to the test compound can be isolated. Because samples (mixed cell suspensions) undergo single-cell analysis, isolation of a cell that has been identified as a GOF variant cell permits the subsequent identification and analysis of the ligand target mutation(s) that produced the GOF activity. This can be accomplished by growing the sorted cells and using standard molecular biology techniques to identify the specific variant or mutation present in the GOF variant ligand target that was sorted by this method. The positive selection sort gate will include those cells that show a detectable or threshold response, and exclude those cells that do not show a response.

In certain embodiments, GOF sorting is performed using a flow cytometer in sorting mode (a fluorescence activated cell sorter), and setting sort gate criteria based on the functional response of the variant GOF cells to the ligand. Sorting can occur during a specifically selected time segment of the dynamic response kinetic (e.g., Time Window sorting) or over a broad time kinetic where the time dependent dynamic nature of the response is visualized during the sorting process. In one embodiment, a mixed cell suspension is rapidly mixed with a test compound such that all cells are exposed to the test compound at nearly the same time, and the sample (the test-compound-treated-mixed cell suspension) is introduced into a flow cytometer at a fixed time after exposure to the test compound. Using single cell flow cytometry, the time after exposure to the test compound and the functional response is determined for each cell, with the result that a kinetic profile (time series) of the functional response in one or more distinct cells populations can be extracted from a single sample.

In accordance with another aspect, sorting could be performed on an “end-point” assay wherein the functional response is developed over minutes or hours to days. Depending on the particular assay, cells may be contacted with a test compound and incubated for a selected amount of time before the functional response of the cells is analyzed. It is understood that end-point assays of a rapid response such as Ca²⁺ mobilization or a change in membrane potential may only need minutes or hours, e.g., 1 hour, before the functional response is measured, while end point assays of a slower response such as gene expression may require hours or days before the functional response is measured. In certain embodiments, GPCR responses are measured using intracellular Ca²⁺ mobilization measurements (Ca²⁺ᵢ). As described above, other functional responses can be measured using other fluorescence based measures of signal transduction events including, but not limited to, changes in membrane potential, changes in cytoplasmic or intraorganellar pH, or fluorescence resonance energy transfer (FRET) assays.

The sorting process could involve sorting many GOF cells into a single pool, possibly containing many variant ligand targets. Alternately, the sorting process could involve depositing single cells into individual vessels or microplate wells for the purpose of culturing distinct clonal populations, each containing a single variant ligand target, e.g., as in the STIS expression system described above. In one embodiment, an instrument has the capacity to sort populations, but not single cell clones, in four different directions, which makes it possible to sort from four different populations simultaneously if desired. Combined with the color-coded multiplexing capability described herein, this would allow simultaneous FACS sorting of four different color-coded cell populations into four different corresponding sorted population, whereby each color-coded population may contain different variant ligand target sets. It may be desirable to sort GOF variants while simultaneously performing small-scale screening operations. The latter approach would provide the option of sorting while a relatively small library is being screened, rather than prescreening compound libraries for active molecules and then sorting for GOF.

Identification and Analysis of GOF Mutations.

The isolated GOF variant ligand target-bearing cells are subjected to any of several molecular biology protocols designed to identify and analyze the variant ligand target that is responsible for GOF. This could include, but is not limited to, sequencing of the variant ligand target or PCR-based identification of the variant, or the use of DNA microarrays. In one embodiment, variant GPCR-bearing cells are analyzed to identify the single mutation responsible for observed GPCR GOF in certain variants. This identification may be significantly more complicated if the one variant ligand target per cell ratio has not been accomplish, as expected in the random integration expression system. In these cases, isolation and retransfection of the variant ligand target DNA constructs present in the sorted GOF cells may be necessary to ascertain which variant ligand target was responsible for the observed GOF phenotype.

Ligand-Receptor Modelling

GOF sorting as provided herein can be used to isolate and identify those variant ligand targets and ligands (“GOF ligand target-ligand pairs”) that show enhanced function. These GOF ligand target-ligand pairs are useful in the identification of specific ligand-receptor interactions, based on correlating the GOF effects of a chemical modification in the ligand with a mutation on the receptor, suggesting an interaction between said chemical modification and the residue(s) in the receptor where the GOF mutation occurs. The simplest model for the ligand-receptor interaction is a direct interaction, and the best way to resolve direct versus indirect interactions is to identify several such potential contacts based on GOF mutation, and explore the consistency of results in the context of the structure of the ligand and receptor complex. If the structure of the ligand-receptor complex is not known, computational models are commonly used to provide the structural context for analyzing this data. The identification of multiple contact sites between a ligand and a receptor by GOF sorting as provided herein, enables the development of experimentally validated computational models of ligand-receptor complexes, which can be used to guide the optimization for the ligand for said receptor, as well as related receptors where the same computational modelling techniques can be applied. Thus optical isolation of GOF mutants using variant related ligands facilitates the identification and optimization of highly selective and potent drug candidates. In one embodiment, the physicochemical and structural information provided by each ligand-GOF variant ligand target pair is incorporated into an analysis and modelling protocol that includes a proprietary algorithm to evaluate the free energy of binding for each pair evaluated. These modelling processes allow rapid modification and improvement of the ligand with respect to many parameters.

Features of GOF Sorting

GOF sorting as provided herein has certain important features. First, it utilizes the combination of a diversity of polypeptide structures, achieved through random or directed nucleic acid mutational techniques, with a diversity of test compounds, to develop an information-rich set of mutant (variant) polypeptide and test compound pairs. The combination of variant polypeptides and diverse ligands gives the approach larger combinatorial power, in comparison to approaches that only involve changing the structure of the polypeptide and observing GOF with respect to a single target such as an antigen of clinical interest.

GOF sorting as provided herein uses accurate pharmacological knowledge of the functional response of the test ligand-test variant ligand target pair to identify those polypeptides with enhanced functional responses (GOF) to the test ligand, and then distinguish the pair from other pairs composed of different variant ligand targets and test ligands. This knowledge can be used to set up the conditions that allow the instrumentation to sort or recover only those activated variant ligand target-bearing cells that show prescribed enhanced efficacy properties (GOF). In contrast, some approaches involve mutating the polypeptide receptors to exhibit enhanced function with respect to a particular ligand, but functional response-dependent sorting is not performed to isolate the particular variant ligand target of interest. (Quehenberger et al., 1997, Biochem Biophys Res Comm 238:377-81; Simpson et al., 1999, Mol Pharmacol 56: 1116-1126). In such approaches, the pharmacological properties of specific mutated receptors are simply characterized by classical techniques. Likewise the literature provides examples of sorting mammalian cells based on real-time functional responses, but these are associated with selection from a naturally occurring heterogeneous mixture of cells and not from a pool of diversely mutagenized receptors transfected into the cells. (Dunne, 1991, Cytometry 12:597-601; Ransom et al., 1991, J Biol Chem, 266:11738-11745; Ransom et al., 1991, J Neurochem 56:983-989).

GOF sorting as provided herein focuses on evaluating membrane-bound proteins that are receptors, e.g., GPCRs, rather than immunoglobulins or enzymes. Receptors are expressed on the surface of cells, and are not secreted or shed, as immunoglobuins are. Likewise, receptors do not catalyze chemical turnover of a substrate, as enzymes do. With optimization of immunoglobulins, the goal is to obtain a secreted protein with optimal binding properties to be used as a therapeutic molecule, diagnostic tool or simple reagent. The goal of enzyme optimization is to obtain a protein with superior catalytic properties. In contrast, the purpose of mutating a receptor as provided herein is to derive information to optimize the ligand by gaining knowledge of specific ligand-receptor interactions in terms of the physical and chemical properties of specific residues responsible for ligand binding, and for promoting or failing to promote activation of the receptor's signal transduction pathway. Thus, GOF sorting as provided herein results in development of improved ligands, not superior target receptors or target proteins such as enzymes and antibodies.

In GOF sorting, the test system in which the variant polypeptides are expressed are generally mammalian cells. However, GOF sorting can also be carried out using yeast strains or other suitable eukaryotic or bacterial host cells to express mutated and wild type receptors and other target proteins such as proteases. In addition, GOF sorting can also be carried out using beads to display targets.

In GOF sorting as provided herein, the signal to be monitored during the ligand-receptor interaction is a functional response, often a signal transduction event such as internal Ca²⁺ mobilization. Other signals such as ligand-receptor binding, receptor expression levles, or combinations of these and other signals, can also be monitored using these techniques.

Cell Sorting

GOF sorting as provided herein differs from previous approaches to cell sorting, providing an active mixing step wherein a mixed cell suspension containing a plurality of target receptor variants and, optionally, different wild type receptors, is rapidly mixed with a test compound. This step is followed by an analysis of functional responses within the cell population. For rapid functional responses, e.g., receptor-mediated signal transduction events, the functional responses are analyzed within about 3 to about 60 seconds after the mixing step. The analysis step is generally accompanied by a “sort” decision and a sorting event, where this aspect is supported by cell sorting hardware and software. Mixing and sorting steps can be repeated several times for a plurality of variant ligand target populations and a plurality of test compounds, resulting in a “high throughput” gain of function analysis and sorting. GOF sorting as provided herein addresses multiple ligand target-ligand combinations.

Fluorescence activated cell sorting (FACS) is a suitable approach to cell sorting. Previous disclosures of the use of FACS to sort mutated polypeptides, used molecular evolution techniques to develop large libraries of either immunoglobulins (single chain monoclonal antibodies), enzymes or binding proteins that are directed towards a single target or substrate. The libraries were expressed in a prokaryotic microorganism (e.g., bacteria or yeast) that was to be used as the assay system. A functional assay (e.g., binding or substrate turnover assay) was designed using a fluorescence parameter as the readout signal. The library-expressing cell population was incubated with the fluorescent assay system, and FACS was used to evaluate a large sample of the entire population. In previous disclosures, FACS sorted those cells that exhibited the preferred activity, such as high enzymatic activity or binding, into a tube. Several rounds of sorting were often required to obtain a population that exhibited the preferred level of gain of function or activity. Descriptions of sorting techniques and various microbial expression systems are found in the scientific and patent literature. (Olsen et al. 2000, Curr Opin Biotechnol 11:331-337; Chen et al., 2001, Nat Biotechnol 19:537-542; Daugherty et al. 2000, J Immunol Methods 243:211-227; Georgiou et al., 1997, Nat Biotechnol 15:29-34; Olsen et al., 2000, Nat Biotechnol 18:1071-10741 U.S. Pat. No. 6,180,341, WO9849286A2, WO9310214A1, WO0234886A2, U.S. Pat. No. 5,348,867). While these methods provided a mutated immunoglobulin, mutated modified immunoglobulin, mutated binding protein or mutated enzyme with increased activity towards only their respective ligands (epitope, cognate protein or substrate), there was no use of modifications of the receptors to gain information to further imporve the cogante ligands. This additional step requires identification of the physical and chemical properties of the ligand that are responsible for optimal interaction with the mutated or wild type receptors (immunoglobulin or enzyme).

Although eukaryotic (i.e., yeast) expression systems have also been used to optimize protein activities where endoplasmic reticulum-specific post-translational processing steps are required for proper folding, these efforts were largely focused on optimizing the affinity of single chain immunoglobulin molecules or T cell receptor molecules, are well described. (Boder and Wittrup, 1997, Nat Biotechnol 15:553-557; Kieke et al., 1997, Protein Eng 10:1303-1310; Cho et al., 1998, J Immunol Meth 220:179-188; EP1056883A1, WO9936569A1, WO0148145A2, WO0148145A3, U.S. Pat. No. 6,300,065, U.S. Pat. No. 6,331,391, U.S. Pat. No. 6,423,538, US20020058253A1). These documents disclose attempts to optimize a phenotypic property of the receptor target polypeptide, such as enhanced affinity, but do not disclose expression mutated polypeptide libraries in mammalian cells, and do not measure signal transduction events for phenotypic selection by FACS and do not refer to automated input systems. Patents that describe the use of automated flow cytometry for drug discovery purposes (U.S. Pat. No. 6,242,209, U.S. Pat. No. 6,280,967, U.S. Pat. No. 6,315,952, US20020015664A1) include disclosures of device with sample loops, peristaltic pump and reciprocating valves to sequentially import multiple samples into a flow cytometer. However, these documents do not appear to disclose mixing of cells with compounds (i.e., mixing from two sample sources) is described, nor is sorting described.

Drug Discovery and Drug Lead Optimization

The methods and compositions disclosed herein are useful in drug discovery and drug lead optimization processes in several ways. First, as part of compound screening efforts, the use of variant ligand targets will identify novel compounds that do not productively interact with the wild type receptor. By sorting and isolating those variant ligand targets that exhibit gain of function properties, the structural differences responsible for the GOF properties of the cell expressing the variant ligand target can be determined. Ligands can be modified based on analysis of ligand-GOF receptor interactions using the modelling techniques described above, giving rise to optimized, and sometimes unique, chemical structures. Such novel ‘hits’ will likely be based on unique chemical structural ‘platforms’ that can be modified through medicinal chemistry to also interact with the wild type receptor. The method also has utility in the lead optimization process. After ‘hit’ molecules are identified and evaluated to select leads, the leads need to be optimized for efficacy and receptor selectivity. Gain of function sorting of variant ligand targets for different test compounds will be used to identify amino acid residues that are critical to the interaction between ligand and receptor leading to productive activation and initiation of signal transduction. Knowledge of such residues permits the development of compounds with the correct positioning of functional groups that will optimally interact with the critical residues in the targeted receptor. The ability to identify critical functional residues in other receptors through gain of function sorting will provide utility in efforts to design chemical ligands devoid of the ability to interact with the non-targeted receptors. That is, compound selectivity for a specific receptor can be improved by designing the molecule to not interact with receptors with similar functional residues while also designing it to favor the residues in the target receptor. Since inadequate receptor selectivity and hence reduced disease target specificity and ‘side effects’ frequently limit the utility of drugs, this aspect of the utility of the invention will be extremely valuable.

Practicing GOF Sorting using Fluorescence Activated Analysis and Sorting.

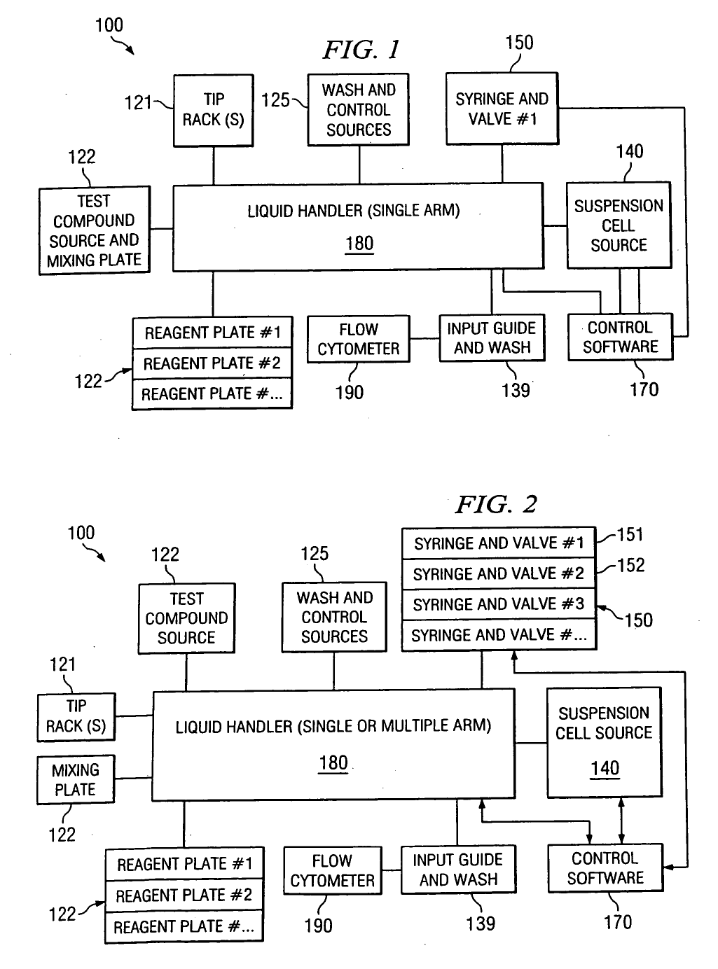

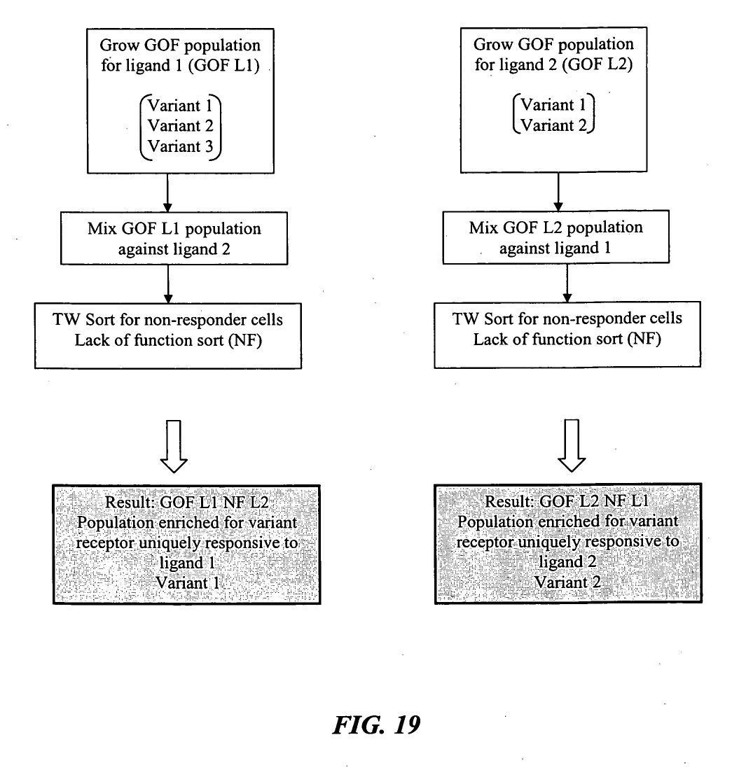

In certain embodiments, GOF sorting uses FACS analysis and sorting of a mutated target protein library expressed in a mammalian cell line, or yeast-based mammalian receptor expression system, where only those cells expressing a receptor with increased affinity for the test compound as compared to the wild type receptor, or another comparison variant ligand target, are detected as initiating a signal transduction event in response to low concentrations of test compound. The overall method is outlined in FIG. 1. The responding cells (gain of function variants) are sorted by the FACS. The invention involves ‘high throughput’ analysis and sorting of a plurality of test compounds and a plurality of variant ligand targets such that a large number of combinations are analyzed and sorted. A detailed description of each step in FIG. 19 is as follows:

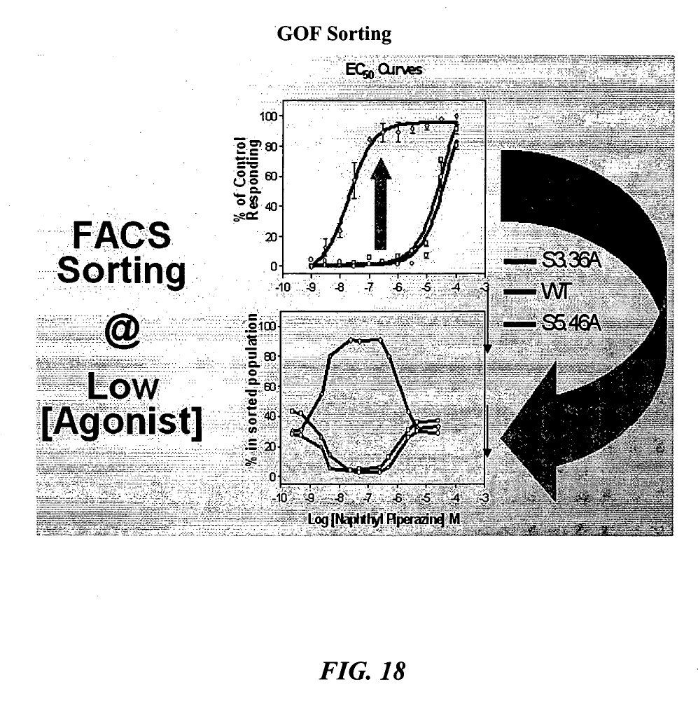

Step 1: To observe GOF behavior it is necessary to understand the range of compound concentration that activates the wild type receptor (WT). GOF variants will respond to concentrations of the test compound at least 5 fold lower than the minimal concentration that activates the WT. An illustration is shown in FIG. 18 where a dose-response curve for one null function mutant receptor (NF), the serotonin 2 A receptor (5 HT2 A) mutant 5.46, and the WT are shown in the lower graph. The two curves are virtually identical. The second mutant (3.36) shows GOF properties since the minimum concentration of the compound that will activate the cells is more than 100 fold lower than that required to activate the WT. The top graph shows how the FACS can sort only those cells bearing the GOF receptor when a mixture of all three receptors are exposed to the compound simultaneously and the sort decision isolates those cells in the mixture that respond to the compound. At very low concentrations, only the GOF 3.36 mutant bearing cells are isolated, and the WT and NF 5.46 mutant are only selected as the compound concentration is raised to relatively high concentrations. It will also be necessary to understand the time kinetics of the WT response so that an appropriate time window (TW) region of the response can be selected for the analysis and sort.

Step 2: FIG. 18 also illustrates that in order to isolate only GOF variants, it is necessary to prepare test compound dilutions that will preferentially activate the GOF variants.

Step 3: For optimal performance it is necessary to limit the number of distinct variant ligand targets within a population to a frequency that is within the resolving limit of the instrument. Deviations from this statistical resolution requirement will result in selection based increasingly on chance events rather than on events influenced by the underlying GOF principle. In cases where the frequency of responding variants is minimally above the resolution of the instrument, cells with non-GOF mutations will be isolated due to instrument error. It will be necessary to perform two or more rounds of GOF sorting to obtain an enriched population of GOF mutants. This process can be improved by using a multiplexing technique so that the percentage of variants in a single resolvable population can reduced, thereby increasing the accuracy of the sorting process and enhancing the purity of the sort, but the total number of variants in the entire analysis will be the same or increased. A method for labeling multiple populations, where each can contain a diversity of variant ligand targets, suitable for multiplexed analysis by flow cytometry, is described in the co-pending patent application entitled “Multiplexed Multitarget Screening Method” filed May 7,2004, the contents of which are hereby incorporated by reference in their entirety.

Step 4: The compounds will be retrieved from the microplates and mixed with cells using an active mixing system as described below. Other systems that bring compounds and cells together have been described but they do not include an active mixing step and this is critical to generating accurate pharmacologic characterizations of the ligand-receptor interaction. Without an active mixing step the potential ligand-receptor interactions do not occur as quickly or efficiently as possible and the ability to clearly resolve GOF variants from NF variants and WT is diminished. The hardware will also operate in an automated manner so that hundreds of test compound-variant ligand target population pairings and comparisons can be iteratively performed in a day.

Step 5: The hardware to be used for mixing will also be capable of introducing the mixed stream of cells and compound into a high speed cell sorter. The sorter may have multiple light excitation sources so that the responses of multiple populations of cells can be analyzed simultaneously and the responding cells sorted out into multiwell plates or one or more collection tubes. The system will inject the mixed cells under a constant time window constraint or may inject the cells so that a dynamic time kinetic is detected. A detailed description of a sample mixing and injection suitable for practicing steps 4 and 5 below, in the section entitled “Direct sample mixing and injection for high throughput fluidic systems,”

Step 6: Following injection of cells into the sorter, the proper gating techniques will be used to collect responding cells from the correct populations, those exhibiting GOF responses, and direct them to the appropriate vessel (e.g., microplate, tube). Responding cells will be defined by visual inspection or by a statistical analysis of unstimulated versus stimulated cells to define the response region that includes a statistically significant proportion of responding cells. The statistical method can be included in this application if appropriate.

Step 7: The cell clones or populations sorted in the first pass can be grown for further functional analysis (FIG. 19, 20) or molecular analysis to determine the variant ligand target insert giving rise to the GOF property.

Extended GOF Sorting using Two Test Compounds

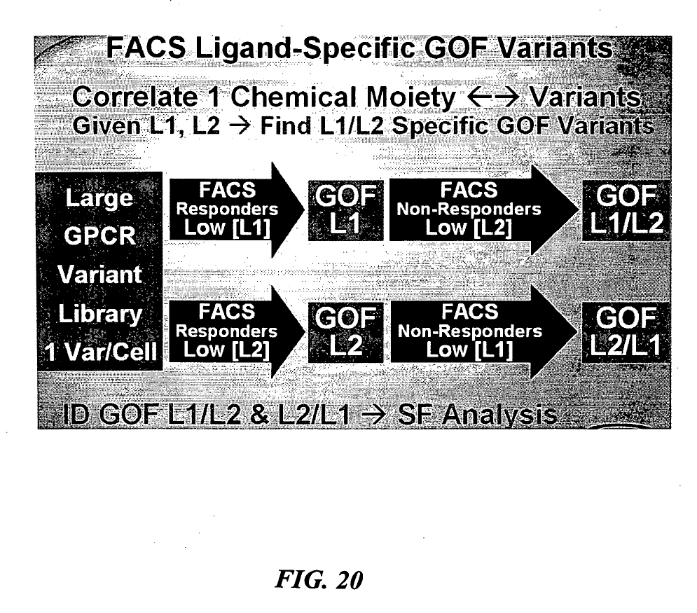

The invention also includes extended (secondary) analysis of GOF variant ligand targets. Extended GOF sorting is described in Example 3 and illustrated in FIGS. 19 and 20. In extended GOF sorting, one or more populations selected for GOF activity towards a first ligand (L1) and no GOF activity towards a second ligand (L2) that is different from the one used to isolate the L1 GOF population, and cells that fail to respond to L2 (non-responding cells) are sorted and isolated. The process can then be run to select for GOF towards the second ligand (L2) by first sorting for GOF towards the second ligand and then sorting for non-responding cells towards the first ligand (L1) as shown in FIG. 20. The non-responding cells sorted in the evaluation against the second ligand are termed “null function mutations” (NF) with respect to the second test compound. Extended GOF sorting provides a rapid means to optimize understanding of the key functional groups on test compounds responsible for triggering the greatest GOF behavior in the variant ligand target population. A detailed description of each step follows:

Step 1. The GOF variant population (GOF L1) selected against compound (ligand) 1 as described above is compared with the GOF variants from compound 2 (GOF L2). The pool of unselected variants used to prepare both GOF L1 and GOF L2 will be the same. GOF L1 and GOF L2 will be expanded so that they can be retested for GOF and NF activity against the alternative ligand. If GOF L1 contains variants 1, 2 and 3 and GOF L2 contains variants 1, 2 and 3 also, and L1 selectively activates Variant 1 while L2 selectively activates variant 2 then selecting those cells that show NF (fail to respond) properties against the converse ligand will yield a population obtained from GOF L1 that is enriched for the one variant ligand target that is unique to ligand 1, variant 1. The converse configuration will yield a population uniquely enriched in variants responsive to L2, variant 2. This method will enhance the resolution of subsets of variant-compound pairings that yield the greatest degree of GOF activity, hence the greatest insight into the molecular basis of the reason for the differences and the greatest information regarding the critical points for functional ligand-receptor interaction.

Step 2. The process is repeated for all GOF populations and compound pairings that have been empirically determined to be of interest, resulting in tens to hundreds of such pairings. This information is used to develop extremely selective drug candidate compounds.

The libraries may be generated by creating one mutant at a time (e.g., Quick Change, Stratagene), or by a random mutagenesis technique (e.g., error prone PCR). Libraries are inserted into an appropriate vector that allows control of protein expression as regulated by a promoter endogenous to the transfected cell, by a constitutively active promoter contained in the vector or by an ‘inducible’ promoter system contained within the vector construct. Libraries and vectors are constructed such that one mutated or wild type target DNA is expressed per cell in the population. The result is a population of cells where each cell expresses only one of the variant targets but each variant in the target DNA library is expressed with a random distribution frequency throughout the entire population. Target proteins to be evaluated/studied include, but are not limited to G-protein coupled receptors, ion channels and single transmembrane receptors. The target proteins must be capable of initiating ligand-dependent signal transduction events that can be monitored as fluorescent signals. Examples of fluorescent signals include Ca²⁺ᵢ mobilization as detected by intracellular Ca²⁺-sensing fluorescent dyes or FRET (fluorescence resonance energy transfer)-based systems that indicate intermolecular distances between polypeptides or domains of a single polypeptide. The cell population expressing the library of mutant proteins is exposed to and thoroughly mixed with a test compound. Each test compound is one of a library of test compounds. The test compounds can be ‘randomly’ synthesized through, for example, a combinatorial chemistry process or synthesized in a directed and focused manner by traditional medicinal chemistry methods.

Direct Sample Mixing and Injection for High Throughput Fluidic Systems

In one exemplary embodiment, the present invention is practiced using a system and method of mixing and injecting discrete sample mixtures into a flow cytometer or other sample analysis apparatus, as described below and illustrated in FIGS. 1-16. In accordance with some exemplary embodiments, for example, a sample injection guide may couple a liquid handling apparatus with a sample analysis apparatus, facilitating injection of discrete sample mixtures into a fluidic system of the apparatus.

As set forth in more detail below, a sample analysis system may generally comprise: a liquid handling apparatus operative to prepare a discrete sample mixture; a sample analysis apparatus; and an injection guide coupled to the analysis apparatus; the injection guide operative to receive the discrete sample mixture from the liquid handling apparatus and to provide the discrete sample mixture to a fluidic system of the analysis apparatus. In accordance with some embodiments, the injection guide may comprise: a guide well operative to engage a pipette tip manipulated by the liquid handling apparatus; and a port in fluid communication with the guide well and operative to receive the discrete sample mixture from the pipette tip and to communicate the discrete sample mixture to the fluidic system. The guide well and the port may be in continuous fluid communication with the fluidic system.

Turning now to the drawing figures, FIG. 1 is a simplified block diagram illustrating functional components of one embodiment of a sample analysis system incorporating elements of a direct sample injection system, and FIG. 2 is a simplified block diagram illustrating functional components of another embodiment of a sample analysis system incorporating elements of a direct sample injection system.

The functional description set forth below is primarily directed to operational characteristics of the FIG. 2 embodiment which may employ a dual pipetting arm liquid handler arrangement, though a single pipetting arm arrangement, such as illustrated in FIG. 1, may also have utility in various applications. Those of skill in the art will appreciate that a sample analysis system as contemplated herein may be susceptible of numerous alterations and modifications, and that the particular configuration of structural components may be selectively adjusted in accordance with myriad considerations including, but not limited to: overall system requirements; size or scale limitations of one or more structural elements; implementation, programming instructions, and computational bandwidth of various processing components; desired sample throughput rates; and other factors. In particular, the present disclosure is not intended to be limited by the number of articulated arms employed by any particular liquid handler apparatus.

As illustrated in FIGS. 1 and 2, an exemplary sample analysis system 100 generally comprises an analysis apparatus such as a flow cytometer 190, for example, and a liquid or sample handling and injection system, such as liquid handler 180. As contemplated herein, references to “direct sample injection” and similar terms are generally related to a process of delivering discrete sample mixtures from liquid handler 180 to an independent fluidic system such as may be incorporated or integrated in a sample analysis apparatus (e.g., flow cytometer 190); it will be appreciated that, in this context, the term “independent” generally refers to a fluidic system of a sample analysis apparatus that is distinct from, or not necessarily integrated with, the structure (in general) and the fluidic system (in particular) associated with liquid handler 180, though used in conjunction therewith in system 100.

In some embodiments, flow cytometer 190 may be implemented in fluorescence activated cell sorting (FACS) applications; additionally or alternatively, flow cytometer 190 may be employed in any of various sample analysis applications generally known in the art or developed and operative in accordance with known principles. In alternative implementations of system 100, flow cytometer 190 may be supplemented or replaced by any of various different types of sample analysis apparatus benefiting from direct sample injection functionality as set forth in more detail below. For example, one such alternative apparatus may include suitable structural elements allowing or enabling various microfluidic applications; those of skill in the art will appreciate that a direct sample injection system may have utility in numerous environments with minimal or no modification.

During use, liquid handler 180 may be operative (under microprocessor or computer control, for example) to prepare samples to be analyzed and to deliver sample material or other liquid mixtures to a flow cytometer 190 or another sample analysis apparatus through a sample injection guide component 139. In that regard, liquid handler 180 in the FIG. 2 arrangement may be embodied in or incorporate any of various commercially available, computer or microprocessor controlled, dual arm liquid handling stations such as, for example, a Cavro RSP 9000 unit; similarly, the FIG. 1 liquid handler 180 may be embodied in or comprise any single arm liquid handling station such as may be generally available or as may be developed and operative in accordance with the functional characteristics set forth herein.

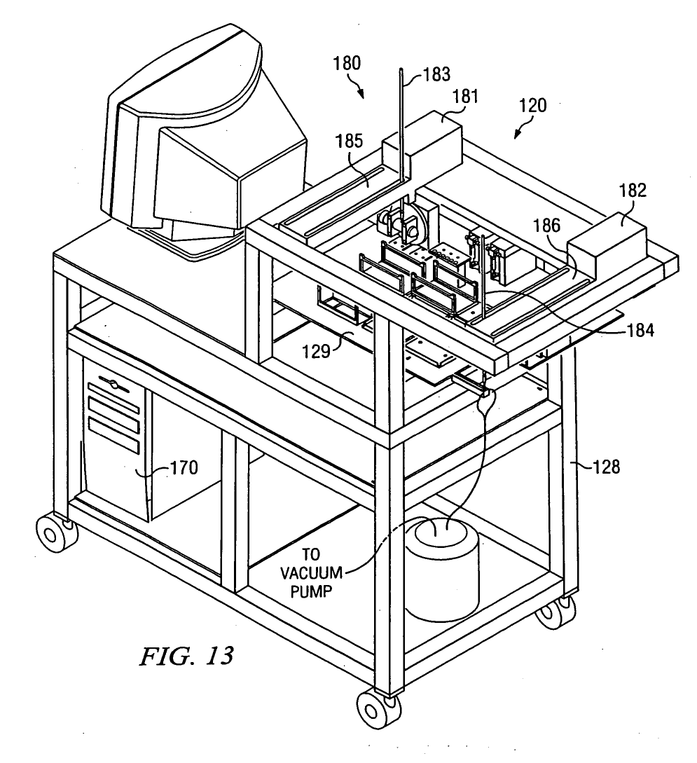

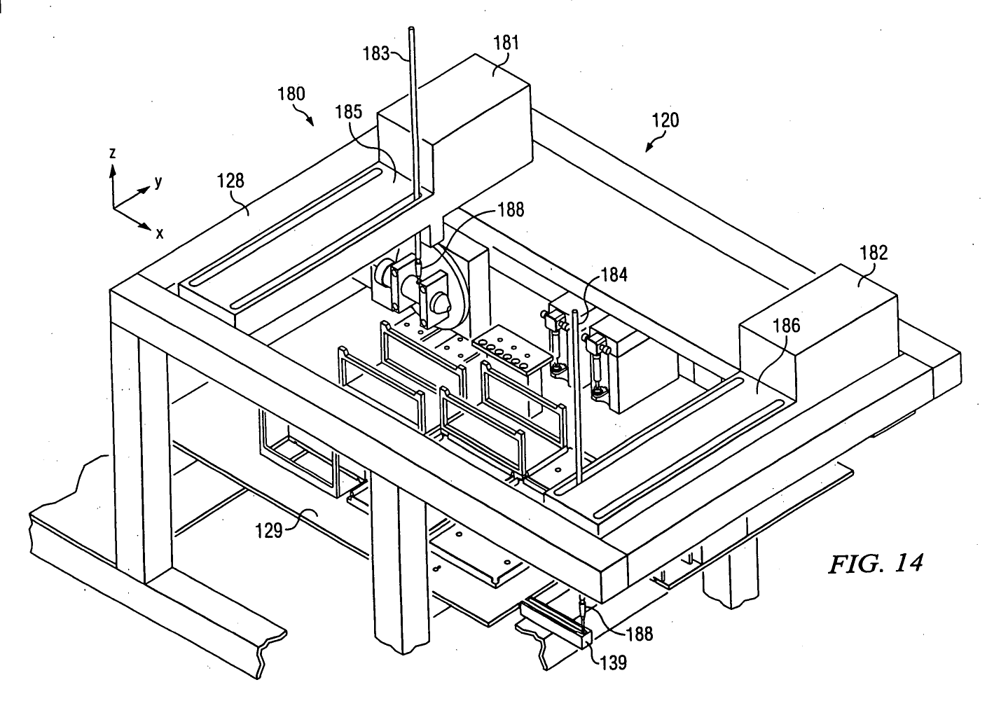

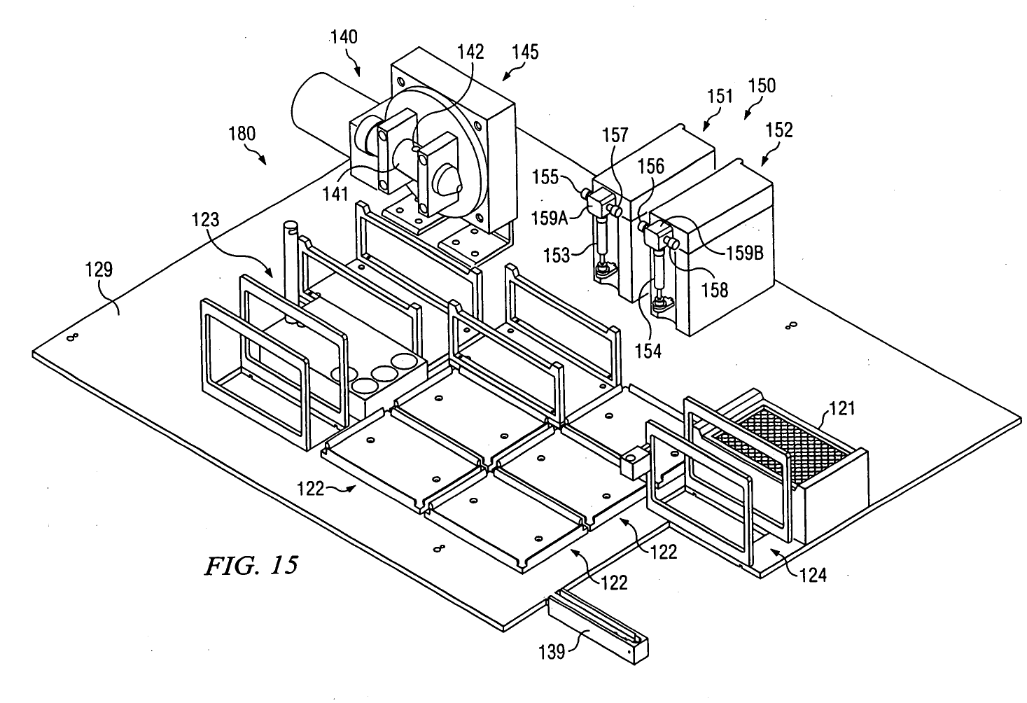

With reference now to FIGS. 13-15 in addition to FIGS. 1 and 2, it is noted that FIG. 13 is a simplified perspective diagram illustrating components of one embodiment of a sample analysis system incorporating a direct sample injection system, FIG. 14 is a simplified perspective diagram illustrating components of one embodiment of a direct sample injection system, and FIG. 15 is a simplified perspective diagram illustrating additional components of the direct sample injection system of FIG. 14.

Liquid handler 180 may generally be configured and operative to implement disposable pipette tips on any number of pipetting arms; as set forth above, while the exemplary embodiment of FIGS. 2, 13, and 14 employs two pipetting arms (reference numerals 181 and 182), systems incorporating one arm (FIG. 1), as well as systems incorporating more than two arms, are also contemplated. Such systems employing an arbitrary number of pipetting arms may be implemented in accordance with the principles and functional attributes described herein. In the exemplary system 100, a respective pipetting probe 183,184 may be suspended from a respective translational support structure 185,186 associated with each respective arm 181,182. Such pipetting arm assemblies accommodate rapid, precise movement of probes 183,184 in x, y, and z (i.e., Cartesian) coordinate directions. For many applications, translation in approximately 0.003 inch (0.076 mm) increments in a particular coordinate direction may readily be achieved using conventional automated or microprocessor controlled liquid handlers; such precision may be sufficient, but may not be necessary, for typical uses. It will be appreciated that the degree of precision with which a pipetting arm (181,182) and its associated support structure (185,186) and probe (183, 184) are moved may be a function of various factors; the present disclosure is not intended to be limited by parameters affecting accurate and precise placement of structural elements in traditional liquid handling systems.

Pipetting arm 181,182, structure 185,186, and probe 183,184 combinations are generally operative to manipulate probes 183,184 in three-dimensional space, enabling probes 183,184 selectively to engage a pipette tip (reference numeral 188 in FIG. 14) which may be fabricated of plastic, acrylic, latex, or other suitable materials as generally known in the art. In that regard, probe 183,184 may be lowered into a rack of pipette tips (reference numeral 121) for coupling of probe 183,184 with a cooperating pipette tip 188. Some such pipette tips 188 currently available may have, for example, a fluid volume capacity of about 20-1000 μl (e.g., Tecan Genesis tips, from VWR/Quality Scientific Products, are available in the foregoing capacity range, and may be suitable for various applications involving automated or semi-automated pipetting procedures).

In some embodiments, a coupling structure or component may facilitate coupling of probe 183,184 with a particular type of pipette tip 188 having known structural dimensions. Specifically, FIGS. 6, 7, and 8 are simplified diagrams illustrating perspective, side elevation, and axial views, respectively, of one embodiment of a coupling component allowing a pipette probe to engage a pipette tip. As illustrated in FIGS. 6-8, a coupling component 110 may generally comprise a conduit 112 through which fluid may be communicated. Coupling component 110 may be fabricated of plastic (such as DELRIN™ for example), acrylic, metal, or other material having suitable strength, rigidity, and corrosion resistance characteristics, for example, which may be application-specific.

Coupling component 110 may comprise an appropriate structural element configured and operative to secure coupling component 110 to probe 183,184; specifically, probe 183,184 and coupling component 110 may be sealingly engaged, preventing leakage or other liquid loss at the juncture therebetween. In the exemplary embodiment, structural coupling or interconnection between probe 183,184 and coupling component 110 is represented as effectuated at a threaded portion 111. It will be appreciated, however, that coupling of probe 183,184 and coupling component 110 may be achieved using other structural elements such as, for example, a quick-disconnect mechanism, a hose barb, or other coupling device having utility in fluidic systems.

Similarly, coupling component 110 may additionally comprise an appropriate structural element configured and operative to secure pipette tip 188 to coupling component 110; as with the connection set forth above, coupling component 110 and pipette tip 188 may be sealingly engaged, preventing leakage or other liquid loss at the juncture therebetween. In the exemplary embodiment, structural coupling or interconnection between coupling component 110 and pipette tip 188 is represented as effectuated at an angled portion 114 operative (e.g., like a hose barb) to engage, under pressure, a cooperating open end of pipette tip 188 having a correspondingly angled inside diameter dimension as generally known in the art. It will be appreciated that coupling of pipette tip 188 and coupling component 110 may be achieved using other structural elements having utility in fluidic systems. In some embodiments implementing automated liquid handling apparatus and techniques, coupling component 110 may additionally allow or enable automated ejection (i.e., disengagement or decoupling) of pipette tip 188 from angled portion 114.

During pipetting operations when coupling component 110 is interposed between probe 183,184 and pipette tip 188, liquid may be communicated from probe 183,184 into conduit 112, and vice-versa, at end 115; similarly, liquid may be communicated from conduit 112 to pipette tip 188, and vice-versa, at end 113. It will be appreciated that the various elements, in general, and the specific structural arrangement, in particular, of coupling component 110 may be susceptible of various modifications, and that aspects of the exemplary structure depicted in FIGS. 6-8 may be selectively dimensioned, altered, omitted, or rearranged in accordance with numerous considerations including, but not limited to, the dimensions and other structural characteristics of probes 183,184, pipette tip 188, or both. For example, where probes 183,184 and pipette tip 188 are suitably constructed for direct coupling or other unassisted engagement, it may be possible to omit coupling component 110 from the fluidic path (i.e., coupling component 110 may not be required for proper operation of some embodiments of liquid handler 180).

As illustrated in FIGS. 1 and 2, a sample analysis system 100 may generally comprise a pump system 150 configured and operative to control fluid flow and liquid handling procedures. As indicated in FIGS. 2 and 15, the pipetting function for each respective pipetting arm 181,182 and probe 183,184 assembly may be driven or otherwise influenced by a respective pump system 151,152. In the exemplary implementation, pump systems 151,152 may be embodied in or comprise computer or microprocessor controlled, servo motor driven syringe and diverter valve systems in fluid communication with the interior of probes 183,184 through flexible tubing, for example, or through some other suitable fluidic path or conduit. One exemplary apparatus, the Hamilton PSD3 Servo syringe pump, is commercially available and may be suitable for use in accordance with the present disclosure.

In operation, a syringe motor (not shown in FIG. 15) may receive commands from control software, firmware, or other programming instruction sets; in FIGS. 1, 2, and 13, such control functionality is represented generally by the reference numeral 170. Accordingly, the syringe motor may be instructed selectively to withdraw a syringe plunger (e.g., to load a syringe 153,154) or to advance the syringe plunger (e.g., to expel contents of syringe 153,154). In some systems, a diverter valve 159A, 159B may also receive commands from control software or some other processing and control component 170 (i.e., hardware, firmware, or software). In that regard, diverter valve 159A,159B may be instructed selectively to allow communication of liquids between syringe 153,154 and a buffer supply source (reference numeral 125 in FIGS. 1 and 2), for example, through a port 155,156, or between syringe 153,154 and probes 183,184 through an alternative port 157,158.

The foregoing arrangement allows syringes 153,154 to fill with an appropriate buffer material (such as PBS or HBSS, for instance) or with other chemical or biological reagents, and selectively to drive the fluid contents of syringes 153,154 through the interior (conduit 112) of coupling component 110 and into or through pipette tip 188 as set forth in more detail below. In particular, the volume of material drawn into or dispensed from pipette tip 188 coupled to a respective probe 183,184 may be controlled (e.g., under hydraulic control) by selective operation of respective pump systems 151,152.

The foregoing operation and various other functional characteristics of system 100 may be controlled by processing component 170. In that regard, processing component 170 may be embodied in or comprise one or more computers, microprocessors or microcomputers, microcontrollers, programmable logic controllers, field programmable gate arrays, or other suitably configurable or programmable hardware components. In particular, processing component 170 may comprise hardware, firmware, software, or some combination thereof, configured, appropriately programmed, and operative selectively to control operational parameters or otherwise to influence functionality of components of system 100. It will be appreciated that processing component generally comprises a computer readable medium encoded with data and instructions, these data and instructions causing an apparatus (such as any of the various components of syste, 100, in general, and liquid handler 180, in particular) executing the instructions to perform some or all of the functionality set forth herein.

Parameters which may be affected or controlled by processing component 170 may include, but are not limited to, the following: timing of movement and precise three-dimensional positioning of arms 181,182, support structures 185,186, probes 183,184, and more particularly, some combination thereof; timing and precise control of pump systems 151,152 including syringes 153,154 and valve assemblies 159A,159B, influencing the volume of fluid in pipette tips 188 and the destination thereof; timing and characteristics of mixing operations (as set forth below); sample injection rates through guide 139 and to an independent fluidic system; and other factors.

Accordingly, processing component 170 may be capable of transmitting control signals or other instructions to various other electrical or electromechanical system elements; it will be appreciated that cooperating electrical and mechanical elements (such as motors, servos, actuators, racks and pinions, gearing mechanisms, and other interconnected or engaging dynamic parts, for example) have been generally omitted from the drawing figures for clarity, as have the various electrical connections and wiring therebetween. In that regard, those of skill in the art will appreciate that control signals may be transmitted from, and feedback from various electromechanical components may be received by, processing component 170 in accordance with any of various communication technologies and protocols having utility in interconnecting or otherwise coupling computer peripheral devices and other electronic components. Specifically, devices implemented in system 100 may be coupled to enable uni- or bi-directional data communication using serial or Ethernet connections, for example, or other standards such as Universal Serial Bus (USB) or Institute of Electrical and Electronics Engineers (IEEE) Standard 1394 (i.e., “FireWire”) connections, and the like. In some embodiments, such coupled components may employ wireless data communications techniques such as BLUETOOTH™, for example, or other forms of wireless communication technologies based upon infrared (IR) or radio frequency (RF) signals.

As indicated in FIGS. 13 and 14, an automated pipetting arm assembly 120 including liquid handler 180 may be mounted on a frame 128, allowing pipetting arm 181,182 and probe 183,184 assemblies to address several different stations (e.g., pipette tip rack station 121, a microwell plate station 122, a tube station 123, and a waste bag station 124) selectively positioned or disposed on a deck or platform 129 generally positioned below arms 181,182. Frame 128 and platform 129 may be constructed of metal (such as aluminum or steel, for example), plastic, acrylic, fiberglass, or other suitably rigid material capable of bearing weight of arms 181,182 and other components of liquid handler 180, pump systems 151,152, stations 121-124, and attendant hardware or consumables disposed thereon.

In particular, as noted above, platform 129 may support several selectable stations 121-124. Examples of the stations include, but are not limited to the following: a microwell plate station (such as indicated at 122) for test compounds (drug candidates); a microwell plate station (such as indicated at 122) for mixing the cells and test compounds (drug candidates) where wells may or may not contain dilution buffer or test compounds at the outset; a rack containing tubes (such as indicated at 123) for holding buffers, probes, or test compound standards; waste bag stations (such as indicated at 124) for discarding tips and for expelling priming buffer from probes 183,184; and racks (such as indicated at 121) for holding predispensed trays of pipette tips. It will be appreciated that various other types of stations accommodating different consumables or other items having utility in experimentation may also be included; further, the specific number and orientation of the various stations 121-124 may be altered in accordance with desired system capabilities or application requirements.

As indicated in FIG. 15, platform 129 may additionally support a sample injection guide 139. In that regard, FIGS. 9, 10, 11, and 12 are simplified diagrams illustrating perspective, plan, side elevation, and axial cross-section views, respectively, of one embodiment of a sample injection guide. In some embodiments, guide 139 may be rigidly or fixedly attached to platform 129 or to some other structural element of frame 128. The attachment may be substantially permanent, for example, such as may be achieved by welds, rivets, pressure or heat sensitive adhesives, or other substantially permanent attachment mechanism; alternatively, guide 139may be removably attached to platform 129 or frame 128 such as by screws, bolts, tabs and slots, or other cooperating structural arrangements, for example. It will be appreciated that a removable or adjustable attachment mechanism may provide flexibility for various applications. In some alternative embodiments, guide 139 may be attached, coupled, incorporated, or otherwise integrated into the structure of flow cytometer 190 or other sample analysis apparatus. In such embodiments, it may be desirable to modify or otherwise to adjust the dimensions or relative positioning of platform 129, other components of frame 128, or some combination thereof, to allow engagement of pipette tip 188 with guide 139 as set forth in detail below.

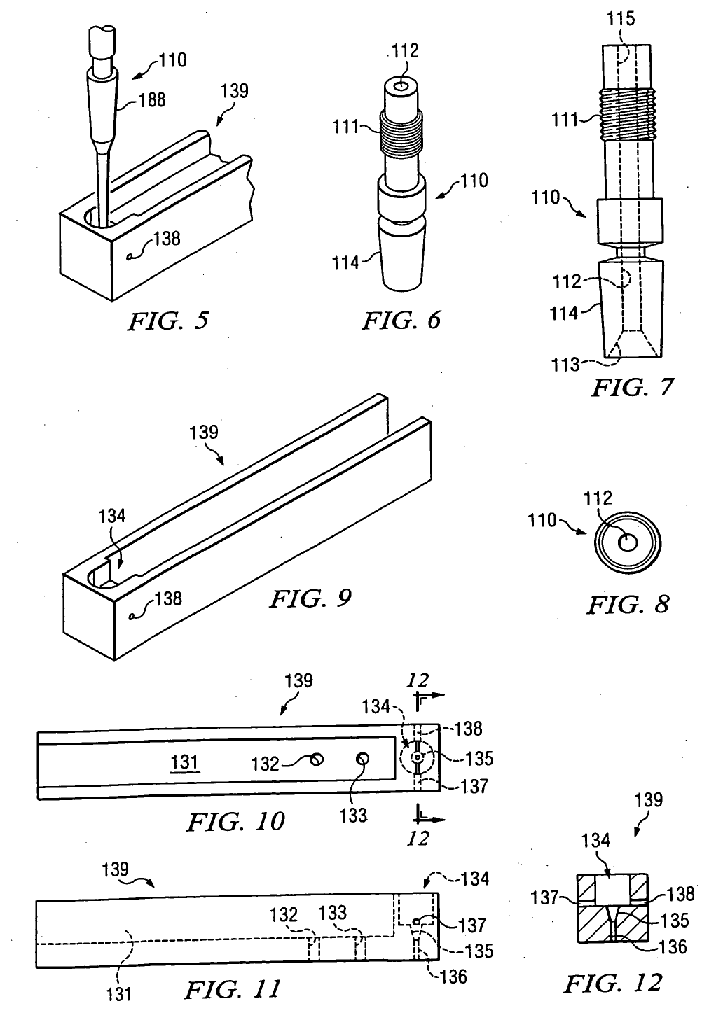

FIG. 5 is a simplified diagram illustrating a perspective view of one embodiment of a sample injection guide engaged with a pipette tip during use. Specifically, guide 139 may be constructed and operative to engage an end of pipette tip 188 and to communicate fluid from pipette tip 188 to the fluidic system of flow cytometer 190 or another sample analysis apparatus. A detailed description of one embodiment of guide 139, as well as some functional characteristics thereof, is provided below.

General Functionality

As set forth in detail above with specific reference to FIGS. 2 and 13-15, functional and mechanical drawings illustrate various components of one embodiment of a sample analysis system 100 employing a dual arm direct sample injection system; the functional attributes of a simpler, single arm embodiment (FIG. 1), as well as those of more complicated embodiments employing more than two pipetting arms, will be readily inferred from the following detailed description of operational characteristics.

Each respective arm 181,182, support structure 185,186, and probe 183,184 assembly may selectively visit tip rack 121 (or a selected, designated, or predetermined one of a plurality of tip racks 121, for example), seal a pipette tip 188 onto the end of each respective probe 183,184, and withdraw the sealed pipette tip 188 in preparation for movement to another station 122-124 on platform 129. As set forth above, probe 183,184 (either in conjunction with coupling component 110 or independently, for example) may form a sufficiently complete seal with pipette tip 188 to allow pipette tip 188 to be withdrawn from tip rack 121 without falling off when probe 183,184 is withdrawn. In particular, such a seal may also be sufficiently complete to prevent air or fluid leakage when fluids are moved into pipette tip 188 from either a reservoir or from a respective pump system 151,152—as described above with particular reference to FIG. 15, pump systems 151,152 may provide fluid (through probes 183,184) and drive volume aspiration and displacement for pipette tip 188.

Coupling component 110 may provide improved sealing between pipette tip 188 and probes 183,184. In one embodiment, for example, coupling component 110 may be fabricated of DELRIN™ plastic, though other plastics, acrylics, fiberglass, and other materials may also be suitable. Coupling component 110 may be constructed to precise dimensional specifications, and may generally be designed and operative to accommodate disposable pipette tips 188 from approximately 20 μl to approximately 1000 μl volume capacity. As set forth above with specific reference to FIGS. 6-8, different disposable pipette tip 188 products may require or substantially benefit from different specifications and structural composition of coupling component 110.

In operation, pipetting arm 182 may be used to inject successive discrete sample mixtures into flow cytometer 190 through guide 139. Initially, arm 182 may position probe 184 at a waste bag station 124, or at some other designated or selected waste vessel location; the attached pipette tip 188 may then be filled entirely (i.e., until a small excess amount is expelled as waste) with working liquid (e.g., buffer). In some embodiments, a desired buffer solution may be drawn through port 156 from a buffer reservoir (reference numeral 125 in FIGS. 1 and 2) into syringe 154. As set forth above, the selective connectivity of syringe 154 with buffer reservoir 125 or the pipette fluid path (via ports 156,158, respectively) may generally be controlled by valve 159B in line with syringe 154; accordingly, the contents of syringe 154 may then be provided to probe 184 and pipette tip 188 through port 158. Filling pipette tip 188 entirely with buffer may remove compressible air bubbles from pipette tip 188 and prevent a discrete sample mixture from being displaced back up into pipette tip 188 during later operations, for example, upon engagement of tip 188 with guide 139 when positive pressure from the fluidic system of flow cytometer 190 communicates with the contents of pipette tip 188. In some simplified dual arm liquid handling embodiments, arm 182 may be used strictly for retrieving discrete sample mixtures from selected locations on platform 129 and successively injecting these discrete sample mixtures into flow cytometer 190 or another analysis apparatus.

In coordinated or substantially simultaneous operations, pipetting arm 181 may also have buffer fluid within the tubing path (i.e., through probe 183 and to pipette tip 188). As described above with specific reference to arm 182, this fluid flow may be regulated through selective operation of syringe 153 and valve 159A of pump system 151. Such buffer fluid may facilitate reduction of compressible air in the tubing path of arm 181. In embodiments where probe 183 of arm 181 does not communicate with the high pressure fluidic system of a sample analysis apparatus (i.e., does not couple or engage pipette tip 188 with guide 139), the buffer solution may not be required to fill pipette tip 188. In the exemplary dual arm liquid handling embodiments, arm 181 may be employed to retrieve cell samples from a cell suspension system (described below) and to dispense these samples into an assay or microwell plate at a selected station 122 on platform 129, to retrieve test compounds (drug candidates) or buffer solution from one or more additional stations 122 at predetermined locations on platform 129 and to dispense same into an assay or microwell plate at a specific station 122 on platform 129, and to perform mixing functions (e.g., mixing the cell samples with compounds, mixing compounds with diluting reagents, or both).

Timing of movements for arm 181 may be keyed off the priorities and movements of arm 182. Specifically, to prevent collisions between arms 181,182, movement conflicts may be resolved, for example, by providing priority to arm 182; in such an embodiment, arm 181 may be required to wait for arm 182 to complete high priority tasks before arm 181 progresses to its next step or location in space. More complicated dynamic prioritization strategies may be employed in sophisticated liquid handling techniques. In the exemplary embodiment employing a strategy in which arm 182 has permanent priority, arms 181,182 may be synchronized to coordinate motions for maximal movement efficiency. It will be appreciated that the particular synchronization strategy employed may be application specific, and accordingly may be affected by the number of samples, compounds, or other reagents to be drawn and dispensed, the number of stations 121-124 in use on platform 129 for a particular application, the number and length of mixing operations to be conducted, the rapidity with which discrete sample mixtures are injected into the analysis apparatus, and other factors.

Arm 181 may address compound plate stations 122 used for agonist mode, antagonist mode, allosteric modulator mode, or various other operational or experimental modalities and protocols. Compounds or reagents may be taken up into pipette tip 188 and added to cell samples or buffer (for dilution purposes) in a predetermined or selected well of a microwell plate at a selected station 122. Mixing of cell sample material and compound or compound and buffer may be performed by arm 181 and probe 183, for example, through selective use of syringe 153 alternatively to draw a mixture from a microwell and to expel the mixture. In some embodiments, a single such cycle may be sufficient to provide adequate mixing, though a mixing cycle may be omitted in some instances, for example, or repeated for any desired number of iterations.

Specifically, arm 181 and probe 183 may address a suspension of viable cell samples and subsequently draw a selected or predetermined sample volume of evenly suspended cells into pipette tip 188 for delivery to a selected well of the microwell plate, i.e., arm 181 and probe 183 may be used to dispense the cell sample volume into microwell plate. Further, arm 181 and probe 183 may be implemented to mix the contents of a specific well (for example, by pipetting up and down a selected or predetermined number of times) without substantially disturbing the cells in the context of the parameters to be measured (e.g., intracellular Ca2+). Alternatively, the injection of cell samples into the well may be sufficient for mixing, eliminating the need for additional pipetting. The cell suspension mixture may then be left in the mixing well until the contents are withdrawn by arm 182 and probe 184 for injection to an analysis apparatus.

After mixing the cell samples and compound for a particular well (i.e., preparing a discrete sample mixture), arm 181 may then travel to waste bag station 124 and automatically eject pipette tip 188 from probe 183. In some embodiments, tip ejection may be monitored, for example, by an IR or other suitable sensor or camera to ensure proper and complete ejection of pipette tip 188. In the case of incomplete ejection, buffer may be rapidly flushed through probe 183 and pipette tip 188, and ejection procedures may be repeated until pipette tip 188 is removed from probe 183. Following confirmation of proper tip ejection, arm 181 may be manipulated to greturn probe 183 to tip rack 121 (or to a different tip rack) to retrieve a new pipette tip 188 in preparation for the next task.

As noted above, arm 182 and probe 184 may withdraw the cell material and compound (a discrete sample mixture) into a pipette tip 188 after an appropriate, predetermined, or otherwise selected duration following mixing; arm 182 and probe 184 may then engage pipette tip 188 with sample injection guide 139 (as illustrated in FIG. 5) and transfer the discrete sample mixture to flow cytometer 190 (or to another sample analysis apparatus).

Regarding injection of discrete sample mixtures into an independent fluidic system, it is noted that FIGS. 9, 10, 11, and 12 are simplified diagrams illustrating perspective, plan, side elevation, and axial cross-section views, respectively, of one embodiment of a sample injection guide. Additionally, as noted above, FIG. 5 is a simplified diagram illustrating a perspective view of one embodiment of a sample injection guide engaged with a pipette tip during use.

Guide 139 and its various components may be fabricated of virtually any suitably non-reactive material. In this context, “non-reactive” generally refers to materials which will not adversely affect the experimentation occurring in the analysis apparatus. In one embodiment, for example, guide 139 may be fabricated of DELRIN™ plastic, though other plastics, acrylics, fiberglass, metals, and other materials may also be suitable.

As indicated in the drawing figures, one embodiment of guide 139 may generally comprise a guide well 135 dimensioned and operative to receive or otherwise sealingly to engage pipette tip 188, and a port 136 in fluid communication with both guide well 135 and the fluidic system of the analysis apparatus. During injection operations, pipette tip 188 may be engaged or seated in guide well 135 such that liquid or air cannot leak through the area of contact between guide well 135 and pipette tip 188. In that regard, it will be appreciated that the general constitution and specific dimensions of guide well 135 (e.g., depth, internal diameter, and taper) may be selected in accordance with the type of pipette tip 188 with which it is intended to be used. For example, guide well 135 is illustrated as tapered in FIGS. 11 and 12; in some embodiments, taper or angular dimensions provided for guide well 135 may be specifically designed to cooperate with a corresponding and complementary tapered portion of pipette tip 188.

When pipette tip 188 is engaged with guide well 135 as set forth above, a discrete sample mixture, or other contents of pipette tip 188, may be injected through port 136 into the fluidic system of the analysis apparatus. Port 136 may be coupled to an independent fluidic system, for example, using flexible tubing, hose barbs, quick-disconnect assemblies, and other types of fluid coupling hardware and mechanisms generally known in the art. This “connection” between port 136 and the independent fluidic system has been omitted from the drawing figures for clarity.

When pipette tip 188 is withdrawn from guide well 135, the free stream dynamic pressure of the independent fluidic system may force liquid back through port 136 and into guide well 135, flushing the connection, port 136, and guide well 135. This flushing may prevent residual material from one discrete sample mixture from contaminating a subsequent discrete sample mixture and altering or otherwise affecting the analysis thereof. It will be appreciated that the dynamic pressure associated with the fluidic system may cause flooding and overflow of guide well 135; additionally, removing liquid back flushed through port 136 into guide well 135 may facilitate minimization of deleterious contamination between successive sample mixtures. Accordingly, some embodiments of guide 139 may additionally comprise an overflow well 134 and siphon ports 137,138.

During operation, back pressure from the independent fluidic system generally causes fluid to flush through port 136 and into guide well 135 and overflow well 134. The depth of fluid in guide well 135 and overflow well 134, on the other hand, may exert sufficient hydrostatic pressure to balance the pressure of the fluid entering wells 135,134 through port 136, preventing a spray or “geyser” effect and minimizing liquid waste. Back flushed liquids (and any sample cells, reagents, or other contamination carried therein) may be siphoned, either by gravity alone, for example, or by pumping mechanisms, through siphon ports 137,138.

It will be appreciated that the structural characteristics, relative dimensions, locations, and orientations of the various elements (i.e., wells 134,135, ports 136-138, and siphon pumps, if implemented) may be selected in accordance with the type of independent fluidic system employed and the operational dynamic pressures expected. For example, an additional siphon port may be required in some instances; alternatively, one or both of siphon ports 137,138 may be omitted. Where no siphon ports are provided, guide well 135 or overflow well 134 may simply be allowed to overflow into a waste drain or bag, for example, or a siphon tube which is not integrated into the structure of guide 139 may be employed.

In the exemplary embodiment, for instance, excess liquid not siphoned from overflow well 134 by siphon ports 137,138 may be directed to a channel 131, where it may then be drained to an appropriate waste container or drain through ports 132,133. Additionally or alternatively, one or both of ports 132,133 may be employed, for example, as guide holes for screws, bolts, or other fastening members, to facilitate attachment of guide 139 to platform 129 or to the analysis apparatus. The present disclosure is not intended to be limited by the structural configuration and design characteristics of guide 139 illustrated in FIGS. 5 and 9-12. It will be appreciated that numerous alterations may be made to guide 139, and that the functionality described herein not limited to the design depicted in the drawing figures.

In accordance with the exemplary embodiment, guide 139 may satisfy the functional requirements set forth below. As best illustrated in FIG. 5, guide 139 may serve as a docking port between a pipette tip 188 containing a discrete sample mixture and an input port (not shown) of flow cytometer 190 or any other sample analysis apparatus employed in conjunction with syste, 100. In the case of flow cytometer 190, for instance, such an input port may be embodied in or comprise a tube in fluid communication with a flow nozzle or cuvette. Guide 139 may have particular utility in cases where hydrodynamic focusing between the discrete sample mixture (injected by pipette tip 188 through guide 139) and sheath fluid in the fluidic system of the analysis apparatus occurs at the input port of the analysis apparatus or just downstream thereof.

In particular, guide 139 may allow the contents of pipette tip 188 to be directly injected through port 136 into flow cytometer 190 (or to any independent fluidic system) on a discrete sample-by-sample basis. Operation of guide 139 enables contents of pipette tip 188 (i.e., a discrete sample mixture) to be treated as, and to behave as, the ideal sample stream described in conventional flow cytometry applications, i.e., where individual sample tubes are manually placed at the sample input station.

Additionally, guide 139 may permit rapid flushing of the sample input tubing (e.g., the input port of the analysis apparatus) to remove adherent compounds and residual sample material from the previous sample mixture. It will be appreciated that the tubing connecting guide 139 (at port 136) to the flow nozzle (i.e., associated with the fluidic system of the analysis apparatus) ideally needs to be washed free of contamination between successive discrete samples; such flushing may prevent sample carryover artifacts in the data stream. To achieve this flushing between successive discrete sample input operations, as set forth in detail above, port 136 and guide well 135 may be in continuous fluid communication with the normal sheath fluid used in the fluidic systems of standard flow cytometers. When pipette tip 188 is disengaged from guide well 135, the sheath fluid of the independent fluidic system (that is normally under positive pressure) washes backwards through port 136. This reverse flow serves to wash the connector tube and the port 136. As set forth above, excess fluid may be removed by gravity, for example, or by continuous aspiration (such as by a vacuum pump) through siphon ports 137,138 and channel 131.

As set forth in detail above, guide 139 may facilitate docking or engagement of pipette tip 188 and guide well 135, allowing pipette tip 188 to be firmly and tightly sealed with the walls of guide well 135; additionally, guide 139 may be operative to prevent the force of docking (i.e., the engagement of pipette tip 188 with guide well 135) from disturbing the alignment between the cells in the sample mixture stream and the lasers of flow cytometer 190 or other equipment in the analysis apparatus. In some embodiments, the foregoing alignment may be achieved by utilizing a length of flexible tubing that communicates sample mixtures from port 136 to the independent fluidic system. Such flexible tubing may absorb stresses associated with repeated engagement of pipette tip 188 with guide well 135, and may prevent transmission of those stresses to components of the analysis apparatus. Maintaining alignment in the foregoing manner may ensure continuous data consistency and quality throughout repeated runs of successive experiments.

Delivery of a discrete sample mixture to the analysis apparatus may be controlled by the pipetting syringe 154 operatively coupled to probe 184 on arm 182 and, in turn, by a motor (such as a servo motor or equivalent device) driving syringe 154. Injection of a discrete sample mixture through port 136 may selectively be rapid and of brief duration, for example, or alternatively, slow and prolonged. In the exemplary embodiment, sample mixture injection rates may be selectively controlled, for example, through control of the servo motor, and thereby the dispense rate of syringe 154. Similarly, pipetting functionality for arm 181 and probe 183, including volumes and rates, may be controlled by a servo-motor driving syringe 153. As set forth above, such control may be effectuated through appropriate programming instructions for processing component 170.

When an injection cycle is completed (i.e., a discrete sample mixture has been injected through guide 139 to an independent fluidic system) arm 182 and probe 184 may move to a waste bag station 124 and eject pipette tip 188 to a waste container substantially as described above with reference to arm 181 and probe 183. As with the foregoing ejection procedure, ejection of pipette tip 188 from probe 184 may be monitored (e.g., by a sensor or camera) to ensure successful ejection of pipette tip 188. Respective arms 181,182 and probes 183,184 may be prepared for the next cycle by retrieving new pipette tips 188 from designated or selected tip racks 121.

In accordance with FIG. 15 embodiment, cell sample material to be analyzed may be maintained in suspension by an active cell suspension system (CSS) 140. During operation, CSS 140 may prevent the cells from settling and, accordingly, may keep cell material at a constant density throughout the entire suspension volume. In that regard, CSS 140 may generally comprise a tube 141 mounted to a rocking apparatus 145. Tube 141 may be loaded with cells and a liquid suspension medium, and generally comprises an aperture 142 allowing access to the contents thereof by pipette tip 188. Tube 141 and its contents may be rocked by rocking apparatus 145 from an horizontal position alternately to positions approximately ±45 degrees off the horizontal axis. In some instances, rocking may be controlled such that CSS 140 does not agitate the suspension in such a manner as to perturb resting cell physiology as measured by fluorescent probes that indicate, for example, Ca2+i membrane potential or plasma membrane integrity.

By way of example, a suspension vessel, such as tube 141, may be a 50 ml sealable plastic tube (e.g., as may be available from Falcon Labware or various other manufacturers), though specific dimensions, volume, and material may be varied as desired. As noted above, tube 141 generally comprises an access port or aperture 142 allowing pipette tip 188 coupled to probe 183 to access the cell suspension in tube 141. In some embodiments, CSS 140 in general, and rocking apparatus 145 in particular, may be under control of processing component 170; responsive to an appropriate control signal from processing component 170, for example, operation of rocking apparatus 145 may be interrupted, and tube 141 may be maintained in a desired orientation, while pipette tip 188 coupled to probe 183 approaches tube 141, enters aperture 142, and withdraws a selected volume of cell sample material. Responsive to an additional signal from processing component 170, or following a predetermined or selected duration, rocking action may be resumed following withdrawal of pipette tip 188 from aperture 142.

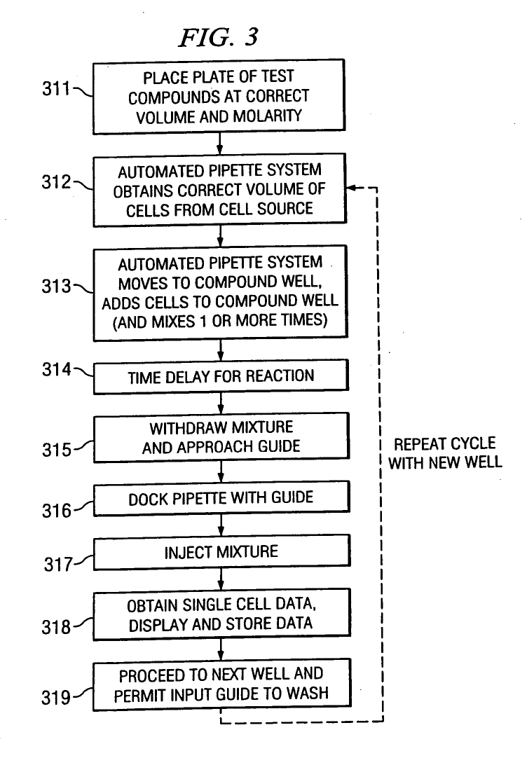

FIG. 3 is a simplified flow diagram illustrating the general operation of one embodiment of a method of performing an analysis using a direct sample injection system. At the initiation of any particular analysis method, as indicated at block 311, a plate of test compounds (at any desired or selected volume and molarity) may be placed at a selected or predetermined station 122 on platform 129; additionally or alternatively, a rack of test tubes, each of which may contain one or more compounds of a selected volume and molarity, may be placed at a selected or predetermined station 123 on platform 129. As set forth above, any number of microwell plates or test tube racks containing various compounds or reagents, or desired combinations thereof, may be placed at one or more such stations 122,123 on platform; specifically, the operation depicted at block 311 may be repeated as desired any number of times and in accordance with a particular analysis protocol. Locations (i.e., at stations 122 or 123 on platform 129) of specific microwell plates or test tubes, as well as the specific contents of each well or test tube and associated data and parameters, may be input or otherwise recorded, for example, using software or other instruction sets, in processing component 170 for further reference, to program sequences of operations executed by arms 181,182 and probes 183,184, and the like.

As indicated at block 312, an automated pipetting apparatus (such as liquid handler 180, for example) may obtain a predetermined or preselected volume of cell material and suspension medium (e.g., from CSS 140). In some embodiments, instructions governing or otherwise influencing the operation depicted at block 312 may be provided by processing component 170 or an equivalent controlling mechanism adapted to provide commands to automated or semi-automated electromechanical systems; additionally or alternatively, such instructions may be provided, in whole or in part, in accordance with user intervention. In the exemplary FIG. 14 implementation, such retrieval of sample cell material may be effectuated by a dedicated pipetting arm 181 and associated hardware, though various other pipetting arm implementations are also contemplated.

Notwithstanding which of a plurality of pipetting arms (such as arms 181,182, for instance) performs the operation at block 312 (or whether a single arm liquid handler 180 is employed), sample material may be added or provided to a specified or predetermined compound well (at station 122) or test tube (at station 123) as indicated at block 313. Specifically, the operation at block 313 represents preparation of a discrete sample mixture (i.e., a mixture comprising a desired volume of sample material obtained from a common sample source (such as from suspension vessel or tube 141, for example) and a specified or preselected compound, reagent, buffer solution, or some desired combination thereof) at a specified location (e.g., at station 122 or station 123) on platform 129. As further indicated at block 313, one or more mixing operations may be conducted. In some instances (depending, for example, upon analysis protocols, the specific chemistry of discrete sample mixtures, and other factors), the foregoing providing sample material to a well or test tube may also effectuate necessary or desired mixing. Alternatively, mixing may be performed through one or more pipetting cycles wherein the discrete sample mixture (of sample material and compound or other chemical components in selected well or test tube) is alternately withdrawn and subsequently returned to the appropriate well or test tube. Again, the operation depicted at block 313 may be influenced or controlled by processing component 170, either automatically or in accordance with user intervention, and driven by a pump system (such as represented by reference numeral 151 in FIG. 15).

As indicated at block 314, a time delay may be provided to allow sufficient time for desired reactions to take place for a particular discrete sample mixture. In some embodiments, such a delay time may be identical, or substantially so, for each discrete sample mixture prepared as set forth above. Alternatively, reaction time durations for one or more discrete sample mixtures may vary from other discrete sample mixtures prepared on platform 129 and awaiting injection into the analysis apparatus. It will be appreciated that synchronization considerations, prioritization strategies, or both, for pipetting arm motions may be influenced or otherwise affected in accordance with the various reaction times required by, or desired for, each discrete sample mixture to be prepared and provided to the analysis apparatus. Accordingly, delay times may be recorded and monitored by processing component 170, for example, and liquid handler 180 may be controlled appropriately to accommodate various reactions and delay durations.

Following a desired or predetermined delay period (block 313) a discrete sample mixture may be withdrawn from its well or test tube station (122 or 123) for delivery or approach to sample injection guide 139 as indicated at block 315. Specifically, each discrete sample mixture prepared in a particular location on platform 129 may be individually addressed and withdrawn successively by liquid handler 180 in accordance with instructions provided, for example, by processing component 170. As illustrated in the drawing figures and described in detail above, an exemplary direct injection system may employ a clean pipette tip 188 for the operation depicted at block 315, eliminating or minimizing contamination between successive injection operations (blocks 316 and 317).

As indicated at blocks 316 and 317, a discrete sample mixture may be injected into the fluidic system of an analysis apparatus substantially as set forth above with specific reference to FIGS. 5 and 9-12. In particular, a pipette tip 188 containing a discrete sample mixture may be docked or sealingly engaged with a sample injection guide 139 (block 316); the discrete sample mixture may then be provided through guide 139 to an independent fluidic system (block 317) associated with a sample analysis apparatus (such as flow cytometer 190). As noted above, an injection rate for a particular discrete sample mixture may be selectively controlled, for example, through operation of a pump system (such as indicated at reference numeral 152) under control of processing component 170.

Data regarding a discrete sample mixture may be recorded, for example, on computer readable media at processing component 170, at another electronic device, or both, for storage or analysis; additionally, such data may be transmitted, via recording media or network data transmissions, for instance, to any desired computerized device or data processing apparatus for recordation or for further analysis. Appropriate, desired, or relevant data relating to the foregoing operations described with reference to blocks 311-315 and 317 may include, but not be limited to, some or all of the following information associated with a particular discrete sample mixture: specific chemistries, volumes, percentages, concentrations, compositions, or other factors related to the discrete mixture of cell samples, compounds, reagents, and buffer solutions; mixing parameters such as the number of pipetting cycles performed, for example, and the forcefulness or rapidity (in terms of fluid flow rates, for example) with which those cycles were executed; the time delay allowed between preparation of the discrete sample mixture and injection of same to the analysis apparatus; the time at which the particular discrete sample mixture is injected into the analysis apparatus, as well as the rate (or duration) of the injection process; and any other parameter monitored or controlled by processing component 170. It will be appreciated that the nature and relevance of data recorded in conjunction with the foregoing processes may be a function of the particular experiment or assay occurring in the analysis apparatus.

Further data may be obtained in accordance with standard or modified operation of the analysis apparatus as indicated at block 318. Though the present disclosure is not intended to be limited to any particular analysis apparatus, or to the operational characteristics or limitations thereof, it is noted that the operation depicted at block 318 may be executed by a flow cytometer 190, for example, or by any other sample analysis equipment known in the art or developed and operative in accordance with known principles of fluidic systems. Data acquired by the analysis apparatus (block 318) may be combined or otherwise associated with the data recorded as set forth above (in conjunction with blocks 311-315 and 317) at processing component 170 or elsewhere; alternatively, separate data files may be maintained for storage or processing as desired.

As indicated at block 319 and the dashed line returning to block 312, the foregoing operations may be executed any number of times, and for any number of discrete sample mixtures sought to be analyzed. As set forth above, processing component 170, or equivalent mechanisms, may be used to record the locations of discrete sample mixtures prepared, and those which have been analyzed versus those that have not.

As set forth above, guide 139 and any attendant coupling tubing or other fluid conduit connecting same to the independent fluidic system may be washed, for example, through a back flush of sheath fluid through operative portions of guide 139. This wash operation, set forth above with specific reference to FIGS. 5 and 9-12, is also depicted at block 319.

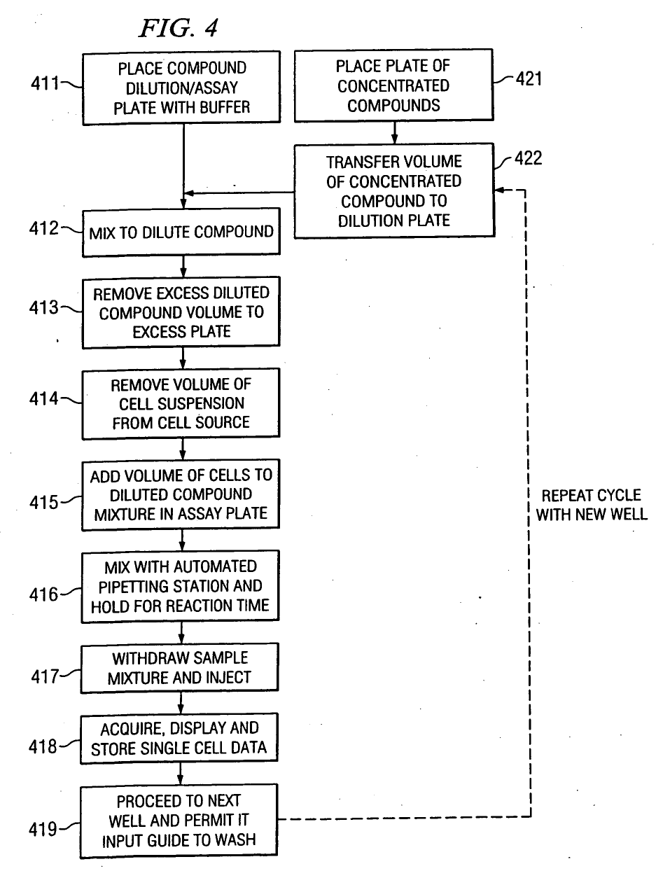

FIG. 4 is a simplified flow diagram illustrating the general operation of another embodiment of a method of performing an analysis using a direct sample injection system. At the initiation of any particular analysis method, as indicated at blocks 411 and 421, various plates or racks of test tubes containing compounds and buffer solutions (at any desired or selected volume and molarity) may be placed at selected or predetermined stations 122,123 on platform 129. As with the method described above, any number of microwell plates or test tubes containing various compounds, reagents, buffers, or desired combinations thereof, may be placed at one or more such stations 122,123 on platform. Appropriate data representative of locations of specific microwell plates or test tubes, as well as the specific contents thereof, may be input or otherwise recorded at processing component 170 or elsewhere. These data may be employed for further reference, to program sequences of operations executed by arms 181,182 and probes 183,184, and the like.

As indicated at blocks 412 and 422, an automated pipetting apparatus (such as liquid handler 180, for example) may transfer one or more compounds to selected other wells or test tubes at specified locations on platform; the resulting combination of liquids may be mixed as indication at block 412. In some embodiments, instructions governing or otherwise influencing the operations depicted at blocks 412 and 422 may be provided by processing component 170 or an equivalent controlling mechanism; additionally or alternatively, such instructions may be provided, in whole or in part, in accordance with user intervention. Mixing at block 412 may proceed substantially as set forth above with specific reference to block 313 in FIG. 3.

Following mixing of desired components, excess liquid may be removed- from a specific well or test tube (block 413) to ensure that the particular well contains an appropriate amount of compound, reagent, buffer, and the like, for creating the desired discrete sample mixture for that particular well or test tube. Excess liquid withdrawn as contemplated at block 413 may be discarded as waste. The operation depicted at block 413 may be selectively controlled in accordance with desired sample analysis protocols for a particular experiment, in whole or in part, by processing component 170.

The operations depicted at blocks 414-416 (i.e., removing or obtaining a desired volume of cell sample material from a source such as CSS 140, for example, adding same to a desired well or test tube, mixing, and allocating a desired delay time), may proceed substantially as set forth above with specific reference to blocks 312-314 in FIG. 3. Specifically, the operations at blocks 414-416 represent preparation of a discrete sample mixture comprising a desired volume of sample material obtained from a common sample source (such as from suspension vessel or tube 141, for example) and a specified or preselected compound, reagent, buffer solution, or some desired combination thereof. This discrete sample mixture may be prepared and maintained at a specified location (e.g., at station 122 or station 123) on platform 129.

As further indicated at block 416, one or more mixing operations may be conducted. Such operations may depend, for example, upon analysis protocols, the specific chemistry of discrete sample mixtures, and other factors substantially as described above. Mixing may not be required in some applications. Further, a time delay may be provided to allow sufficient time for desired reactions to take place for a particular discrete sample mixture. While such a delay time may be identical, or substantially so, for each discrete sample mixture, reaction time delays for one or more discrete sample mixtures may vary from other discrete sample mixtures. Accordingly, synchronization considerations, prioritization strategies, or both, for pipetting arm motions may be influenced or otherwise affected. Where required, one or both of the operations depicted at block 416 may be influenced or controlled by processing component 170, either automatically or in accordance with user intervention.

The operations depicted at blocks 417-419 (i.e., withdrawing and injecting a discrete sample mixture, acquiring data from an analysis apparatus, and reiterating the procedure), may proceed substantially as set forth above with specific reference to blocks 315-319 in FIG. 3. In particular, a discrete sample mixture may be retrieved by liquid handler 180 and injected (block 417) into the fluidic system of an analysis apparatus as described above with specific reference to FIGS. 5 and 9-12. In that regard, a pipette tip 188 containing a discrete sample mixture may be docked or sealingly engaged with a sample injection guide 139; the discrete sample mixture may then be provided through guide 139 to an independent fluidic system associated with a sample analysis apparatus (such as flow cytometer 190). An injection rate or duration for a particular discrete sample mixture may be selectively controlled, for example, through operation of a pump system (such as indicated at reference numeral 152) under control of processing component 170.

Relevant or desired data associated with a discrete sample mixture may be recorded, transmitted, or both, for example, under control of processing component 170 substantially as set forth above. As in the FIG. 3 embodiment, these data may include: specific chemistries, volumes, percentages, concentrations, compositions, or other factors related to the discrete mixture of cell samples, compounds, reagents, and buffer solutions; mixing parameters; the time delay; the time (and rate) at which the particular discrete sample mixture is injected into the analysis apparatus; and any other parameter monitored or controlled by processing component 170. The nature and relevance of data acquired, recorded, or otherwise manipulated in conjunction with the foregoing processes may be a function of the particular experiment or assay occurring in the analysis apparatus.

Additional data may be acquired in accordance with standard or modified operation of the analysis apparatus as indicated at block 418. Finally, as indicated at block 419 and the dashed line returning to block 422, the foregoing operations may be iterated any number of times, and for any number of discrete sample mixtures sought to be analyzed. Processing component 170, or equivalent mechanisms, may be used to record the locations of discrete sample mixtures prepared, and those which have been analyzed versus those that have not. Guide 139 and any attendant coupling or fluid conduit connecting same to the independent fluidic system may be washed, for example, through a back flush of sheath fluid through operative portions of guide 139. This wash operation, set forth above with specific reference to FIGS. 5 and 9-12, is also depicted at block 419.

The specific arrangement and organization of functional blocks depicted in FIGS. 3 and 4 are not intended to be construed as implying any particular order or sequence of operations to the exclusion of other possibilities. Alternative sequences, combinations and simultaneous execution of various operations are also contemplated, and may be enabled or facilitated, for example, in multiple arm liquid handler embodiments and during successive iterations of sample injection cycles. For example, the operations depicted at blocks 315-319 with respect to one sample mixture may occur in parallel, or substantially simultaneously, with operations 312-314 conducted with respect to a different or subsequent iteration for a next successive or different discrete sample mixture. Similarly, the operations depicted at blocks 422 and 412-416 (with respect to one sample mixture) may be executed in parallel, or substantially simultaneously, with the operations depicted at blocks 417-419 (with respect to a sample mixture previously prepared). Those of skill in the art will appreciate that the operations depicted at blocks 317 and 318 may occur substantially simultaneously; similarly, the injection operation (block 417) and the acquisition operation (block 418) depicted in FIG. 4 may also be executed substantially simultaneously.

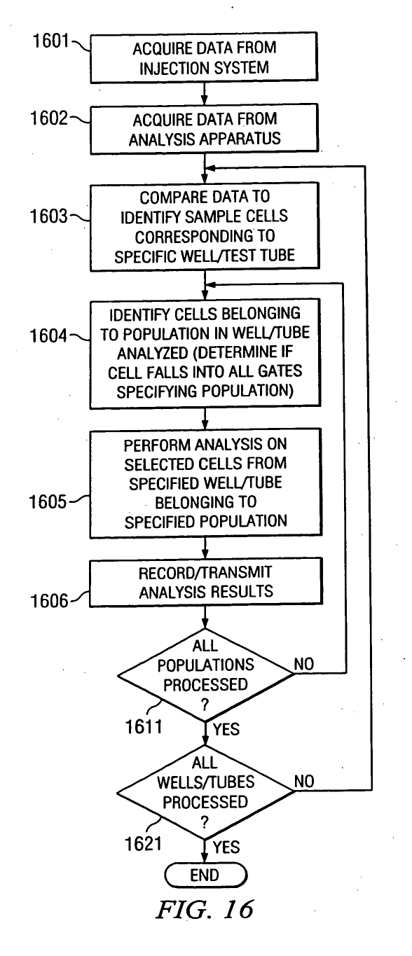

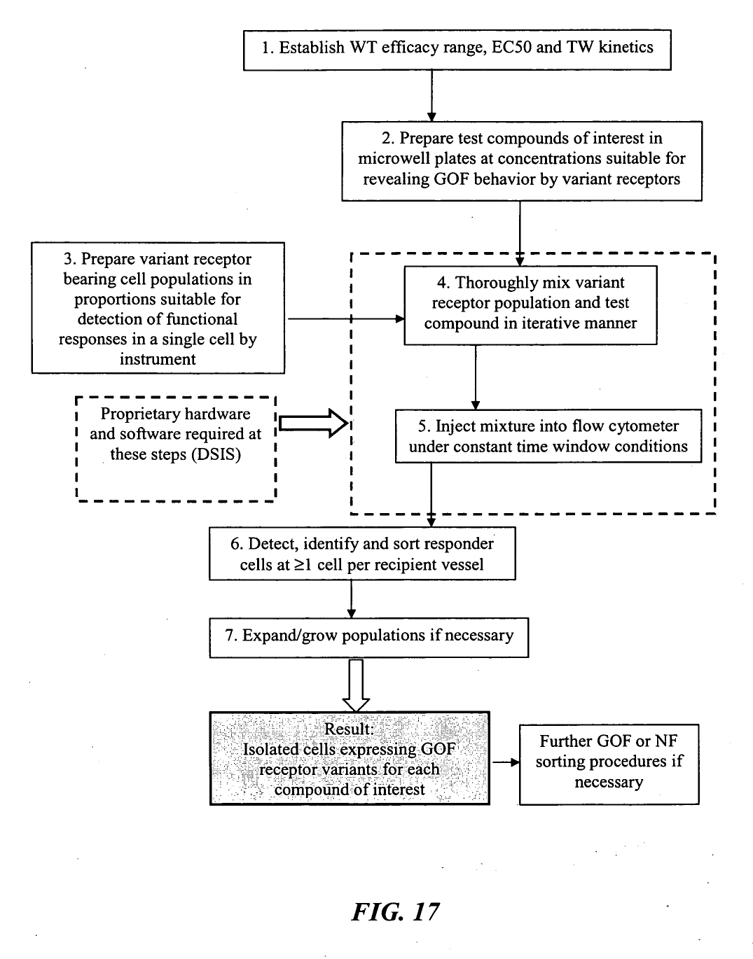

FIG. 16 is a simplified flow diagram illustrating the general operation of one embodiment of a method of performing an analysis. As indicated at blocks 1601 and 1602, data may be acquired from a sample injection system (such as by processing component 170, for example) and from an analysis apparatus substantially as set forth above with specific reference to FIGS. 3 and 4. Acquired data may then be compared (block 1603) to identify which data records obtained by the sample analysis apparatus correspond with data records obtained and recorded by the injection system associated with a particular discrete sample mixture. Where an injection time and rate for a particular sample mixture are recorded by processing component 170, for example, data acquired by the analysis apparatus at that time and for a specific duration thereafter may be flagged as associated with that particular discrete sample mixture. In the foregoing manner, data from the analysis apparatus may be correlated with data from the injection system such that data records may be matched and associated with a specific discrete sample mixture. This correlation may be have particular utility in ascertaining which analysis results are obtained from the sample mixture in a particular well or test tube; in some applications, correlating analysis results with the composition of a sample mixture may facilitate interpretation of the results.

As indicated at block 1604, cell sample material belonging to a particular population may be identified and associated with a specific well or test tube from which the sample mixture was prepared and drawn. In accordance with one embodiment, for example, the identification of cells within a population may comprise determining if a cell falls into all gates specifying the population sought to be identified. It will be appreciated that these gates, and other sorting criteria or parameters, may be user-specified and application specific. In the foregoing manner, cells within a particular well or test tube may be associated with the population criteria appropriate or desired for a particular experiment.

A selected or desired analysis may then be performed on selected cells from a particular well or test tube (i.e., discrete sample mixture) that are identified as belonging to or associated with a particular population as indicated at block 1605. Various analyses including statistical analytical techniques are contemplated at block 1605. For example, mean intensity, median intensity, percentage of cells exceeding a predetermined threshold intensity value, and the like, may be appropriate or desired. It will be appreciated that the nature of the analysis performed at block 1605, as well as the nature of the data records acquired in conjunction with its execution, may vary in accordance with some or all of the following, without limitation: the type of analysis apparatus employed; the functional characteristics and limitations thereof; the operational modality or parameters set to control the analysis apparatus; the type of experiment conducted; and other factors.

Data acquired during the analysis at block 1605 may be recorded, transmitted, processed, or otherwise manipulated as generally indicated at block 1606. Recorded data records may be saved or stored, for example, on computer readable media for processing at a later time; additionally or alternatively, data processing may occur simultaneously or in conjunction with the recordation depicted at block 1606. As set forth above with reference to FIGS. 3 and 4, data may be transmitted via recording media, for instances, or via network data communications to any desired computerized device or processing apparatus.

As indicated by the decision blocks 1611 and 1621, the foregoing process may be selectively iterated, for example, until all populations and all discrete sample mixtures have been analyzed. The iterative nature of the FIG. 16 embodiment may be selectively interrupted in accordance with user intervention if desired.

The entire contents of all references, patent applications, and patents cited in the present disclosure are hereby explicitly incorporated by reference in their entirety.

Aspects of the present invention have been illustrated and described in detail with reference to particular embodiments by way of example only, and not by way of limitation. It will be appreciated that various modifications and alterations may be made to the exemplary embodiments without departing from the scope and contemplation of the present disclosure. It is intended, therefore, that the invention be considered as limited only by the scope of the appended claims.

### EXAMPLES

### Example 1

### GOF Sorting of Variant Human Serotonin Receptor Type 2 A Populations

FIG. 18 illustrates the results obtained during steps of GOF sorting of variant Human Serotonin Receptor Type 2A populations. Three populations of cells containing one of three versions of the human serotonin receptor type 2A were stained three distinct “colors” recognizable by the FACS. The populations were combined in equal proportions into one population. One mutant is the S3.36 A, the other mutant is the S5.46 A mutant and the third group is the wild type receptor (WT). The cells were also loaded with the Ca²⁺ᵢ indicator dye, indol, so that receptor-mediated signaling events could be monitored through the Ca²⁺ᵢ mobilization response. The combined population was stimulated with increasing concentrations of the test compound, naphthyl piperazine, and the percentage of cells within each colored subpopulation showing a threshold Ca²⁺ᵢ response was quantified. The S3.36 A variant ligand target had a greater affinity for the compound than the other two and thus qualified as a GOF variant. At the same time as the analysis was performed, the cells exhibiting the Ca²⁺ᵢ response were sorted into a collection tube. The sorted cells were then re-analyzed to evaluate the subpopulation from which they were derived, based on the distinct “color” used to stain each subpopulation. The identity of the sorted populations that were taken from sorting at several different compound concentrations is shown in the bottom half of the figure. These results validated the basic concept of GOF sorting, since the GOF variant (S3.36 A) was selected away from the other cells at the concentration ranges where only the GOF variant cells were selectively activated by the compound. At the high and low ends of the concentration range there was no selective responsiveness by the S3.36 A variant cells, and the FACS selected each of the populations equally.

### Example 2

### Isolation Gain of Function GPCR Variants by EC₅₀-based FACS Sorting

GOF sorting enhances drug discovery by providing the ability to identify in a few months the Gain of Function (GOF) receptor variants that normally took years to identify. It was hypothesized that GOF variants could be isolated efficiently from a pool of cells, each expressing a single variant, by sorting responding cells at low concentrations of agonist, using Fluorescence Activated Cell Sorting (FACS). The experiment to test this hypothesis focused on GOF variants of the receptor with an enhanced EC₅₀, whose dose response curve was significantly shifted to the left. An example is shown in FIG. 19 for the S3.36 A variant of the 5HT2 A receptor, with a 1,000-fold lower EC₅₀ than the wild type or the S5.46 A variant for the agonist Naphthyl Piperazine. According to these dose-responses, it was hypothesized that after mixing these three 5 HT2 A receptor cell lines and exposing them to low concentrations of agonist, as indicated by the orange arrow in FIG. 19, cells expressing the S3.36 A variant would be selectively activated, and not cells expressing either the wild type or the S5.46 A variant. In fact, FACS sorting of responding cells at this low agonist concentration effectively isolated the GOF S3.36 A variant from a mixture of these 3 5 HT2 A receptor cell lines. To test this hypothesis, each one of the 3 cell lines was “colored” with a different cell tracker dye (Molecular Probes), mixed the three cell lines in a common pool, loaded with the Ca²⁺ sensing dye Indo-1, and repeated FACS sorts were repeated at the different agonist concentrations covering the entire dose-response range. At each concentration, the pool of FACS sorted cells was reanalyzed in the flow cytometer measuring the percentage of each color coded cell line present, which represented the 3 different 5 HT2 A receptor cell lines. This procedure was very efficient in selectively sorting the GOF variant S3.36 A from the wild type and S5.46 A variant in the agonist concentration range 10-300 nM. At lower agonist concentrations, only randomly activated cells were sorted, and thus all 3 cell lines were equally populated. At 3 nM, cells expressing the S3.36 A variant were becoming activated while the WT and S5.46 A variant were hot, and as a result the sorted population was increasingly enriched in the GOF S3.36 A variant until it reached 90% of the sorted population in the 50-100 nM concentration range. At higher agonist concentrations, the WT and S5.46 A cell lines became activated, thus increasing their population percentage in the sorted cells, until all 3 cell lines were equally populated in the sorted population at the highest agonist concentrations sampled. In practical terms, FACS sorting at 100-fold agonist concentrations relative to the WT EC50 selectively isolated GOF variants, and 10-fold lower concentrations also isolated GOF variants but with less discriminating power. It is thus feasible and very effective to isolate GOF variants from a population of variants expressed at one variant per cell by FACS sorting at low agonist concentrations. Either cell populations enriched in GOF variants could be sorted, or single cells that can be grown and their dose-responses evaluated to select a subset of GOF variants for sequencing. More than one pass could be sorted to enhance discriminating power.

### Example 3

### Extended GOF Sorting Using Two Test Compounds

An extended method for GOF sorting is outlined in FIG. 19 and FIG. 20. In this example two ligand test compounds were tested against one population containing two or more variant ligand targets, preferably hundreds to thousands of variants. The goal was to isolate or retrieve those variants that exhibit GOF with respect to ligand 1 (L1) and those with GOF with respect to ligand 2 (L2). The population was first exposed to low concentrations of L1 to obtain those variant cells preferentially responding to L1. The sorted population was retrieved and exposed to L2, and only the cells that failed to respond were removed from the sample by sorting. Thus, the selected population was highly enriched to be highly responsive to L1 as compared to L2. Similarly, L2 was used against the same starting population to select variants responsive to L2, and non-responding cells were removed. This sorted population was then re-evaluated for non-responsiveness to L1 yielding a final population highly enriched for responsiveness to L2 but not L1. The variants were identified by molecular biology techniques, providing information defining the unique properties that differentiate the activity of L1 from L2 at the receptor.

1. A method of Gain of Function (GOF) sorting comprising:
(a) expressing a plurality of variant ligand targets on a plurality of cells;
(b) labelling each cell with a functional target-coupled readout system to monitor a functional response
(c) contacting the plurality of cells expressing the variant ligand targets with a ligand to form a mixture;
(d) analyzing the mixture from step (c) by a single cell analysis system, comprising measuring the functional response of each cell expressing a variant ligand target;
(e) optically isolating individual cells expressing a variant ligand target that shows gain of function (GOF) activity towards the ligand;
(f) identifying the mutation responsible for the GOF activity in each cell isolated in step (e)
wherein the results of (f) are used for designing ligands capable of eliciting an optimal response from a wild type ligand target.

2. The method of claim 1, wherein the majority of cells express one or more variant ligand targets per cell.

3. The method of claim 1, wherein the majority of cells express one variant ligand target per cell.

4. The method of claim 3, wherein homologous recombination is used to express one variant ligand target.

5. The method of claim 4, comprising using the Single Target Integration Site (STIS) for homologous recombination.

6. The method of claim 1, wherein optically isolating cells expressing a variant ligand target that shows gain of function (GOF) activity of a receptor comprises measuring affinity of the ligand for the ligand target, EC₅₀ of the ligand for the ligand target, kinetics of the functional response, level of ligand target expression, or level of functional response.

7. The method of claim 1, further comprising expanding the sorted cells to prepare populations of cells expressing receptors showing GOF activity.

8. The method of claim 1, wherein variant ligand target expression is regulated by an inducible promoter.

9. The method of claim 1, wherein the plurality of cells comprises a cell population expressing a wild type receptor.

10. The method of claim 1, wherein the cells are mammalian, insect, yeast, or bacterial cells.

11. The method of claim 1, wherein the ligand is a chemical or biological ligand.

12. The method of claim 1, wherein the ligand is an agonist, antagonist, neutral antagonist, inverse agonist or an allosteric modulator of the variant ligand target.

13. The method of claim 1, wherein the functional target-coupled readout system is a kinetic or an end-point assay.

14. The method of claim 1, wherein the functional target-coupled readout system is Ca²⁺ mobilization measured by fluorescent indicator dyes.

15. The method of claim 1, wherein the single cell analysis system comprises fluorescence activated cell sorting (FACS), microfluidics-based systems, or microscopy-based systems.

16. The method of claim 15, further wherein the cells are optically sorted by FACS, microfluidics-based systems, or microscopy-based systems.

17. The method of claim 1, wherein the mixture of step (c) is introduced into the sample analysis system by an automated sampling system.

18. The method of claim 17, wherein the automated sampling system is the Direct Sample Injection System (DSIS).

19. The method of claim 18, further wherein the cells are optically isolated by FACS.

20. The method of claim 1, wherein the functional target-coupled readout system comprises measuring expression of the variant ligand target.

21. The method of claim 20, wherein the variant ligand target further comprises an epitope tag.

22. The method of claim 1, wherein a multiplexing technique is used to prepare a mixture of a plurality of resolvable cell populations expressing variant ligand targets.

23. The method of claim 1 wherein the functional target-coupled readout system measures fluorescence, fluorescence polarization, fluorescence lifetime, incident light scatter, electromagnetic field induction, light absorbance, luminescence, fluorescence resonance energy transfer (FRET), bioluminescent resonance energy transfer (BRET), or cell morphology.

24. A method of ligand-selective GOF sorting, comprising:
(a) performing steps (a) through (e) of claim 1 using a first ligand (L1) to isolate a population of L1 GOF cells expressing variant ligand targets that show GOF activity towards L1;
(b) performing steps (a) through (e) of claim 1 on L1 GOF cells of step (a) using a second ligand (L2) to isolate a second population of cells expressing variant ligand targets that do not show GOF activity towards L2, and
(c) identifying the mutation in the variant ligand target responsible for the L1 GOF activity and lack of L2 GOF activity of cells isolated in step (b),
wherein the variant ligand targets of the cells isolated in step (b) are ligand-selective GOF variant ligand targets for L1 with respect to L2.

25. A method of Gain of Function (GOF) sorting comprising:
(a) expressing a plurality of variant ligand targets on cells, wherein the variant ligand targets are naturally occurring variant ligand targets;
(b) labelling each cell with a functional target-coupled readout system to monitor a functional response;
(c) contacting the plurality of cells expressing the variant ligand targets with a ligand to form a mixture;
(d) analyzing the mixture from step (c) by a single cell analysis system, comprising measuring the functional response of each cell expressing a variant ligand target;
(e) optically isolating individual cells expressing a variant ligand target that shows gain of function (GOF) activity towards the ligand; and
(f) identifying the mutation responsible for the GOF activity in each cell isolated in step (e)
wherein the results of step (f) are used to predict drug responses for patients.

26. The method of claim 25, wherein the naturally occurring variant ligand targets are single nucleotide polymorphisms (SNPs).

27. The method of claim 25, wherein step (d) comprises measuring loss of function, such that the optically isolated cells express loss of function variant ligand targets.

28. The method of claim 27, wherein the loss of function is antagonism or inverse agonism.

29. The method of claim 25, further comprising optically isolating cells that do not show GOF activity toward a ligand.

30. The method of claim 29, further comprising iteratively repeating steps (c) through (e) using increasing concentration of the ligand in each iteration, wherein cells that do not show GOF activity toward the ligand at a first concentration are optically isolated and screened using steps (c) though (e) at a higher concentration of the ligand, whereby functionally significant alterations of the effect of the drug on naturally occurring variant targets are identified.

31. A database comprising results generated by practicing the method of claim 25.

32. A database comprising results generated by practicing the method of claim 27.

33. A database comprising results generated by practicing the method of claim 30.
